(19) United States
(12) Patent Application Publication
(10) Pub. No.: US 2015/0250663 A1
(43) Pub. Date: Sep. 10, 2015

(54) THREE-DIMENSIONAL SUBSTRATES

(71) Applicant: The Procter & Gamble Company, Cincinnati, OH (US)

(72) Inventors: Jason Ashley WAGNER, Lawrenceburg, IN (US); Amanda Margaret BICKING, Cincinnati, OH (US); Jörg ENDRES, Frankfurt Am Main (DE); Olaf Erik ISELE, West Chester, OH (US); John Joseph LITCHHOLT, Lawrenceburg, IN (US); Sue Ann MILLS, Cincinnati, OH (US); Rachael Eden WALTHER, Union, KY (US); Paul Thomas WEISMAN, Cincinnati, OH (US)

(21) Appl. No.: 14/635,001

(22) Filed: Mar. 2, 2015

Related U.S. Application Data
(60) Provisional application No. 61/948,702, filed on Mar. 6, 2014.

(51) Int. Cl. A61F 13/511

(52) U.S. Cl. CPC A61F 13/51104

(57) ABSTRACT

A three-dimensional, liquid permeable substrate for an absorbent article is disclosed. The liquid permeable substrate comprises a first layer and a second layer joined to the first layer to form a composite. The composite comprises a plurality of apertures defined therein, a plurality of bridge portions, a plurality of first projections extending outwardly relative to the bridge portions and a plurality of second projections extending outwardly relative to the bridge portions and the first projections. A majority of the plurality of the second projections have a first z-directional height and a majority of the first projections have a second z-directional height. A majority of the plurality of the bridge portions have a third z-directional height. The first z-directional height may be greater than the second and third z-directional heights. The substrate has an overall z-directional height in the range of about 1300 μm to about 2500 μm.

### CROSS REFERENCE TO RELATED APPLICATION

This application claims priority under 35 U.S.C. §119(e) to, U.S. Provisional Patent Application Ser. No. 61/948,702, filed on Mar. 6, 2014, the entire disclosure of which is fully incorporated by reference herein.

### FIELD

The present disclosure is generally related to three-dimensional substrates, and, is more specifically related to, three-dimensional substrates for absorbent articles and/or to absorbent articles comprising three-dimensional substrates. The three-dimensional substrates may comprise liquid permeable substrates.

### BACKGROUND

Absorbent articles for personal hygiene, such as disposable diapers for infants, training pants for toddlers, adult incontinence undergarments, and/or sanitary napkins are designed to absorb and contain bodily exudates, in particular large quantities of urine, runny BM, and/or menses (together the “fluids”). These absorbent articles may comprise several layers providing different functions, for example, a topsheet, a backsheet, and an absorbent core disposed between the topsheet and the backsheet, among other layers, if desired.

The topsheet is generally liquid permeable and is configured to receive the fluids being excreted from the body and aid in directing the fluids toward an acquisition and/or distribution system and/or towards the absorbent core. In general, topsheets are made to be hydrophilic via a surfactant treatment applied thereto so that the fluids are attracted to the topsheet to then be channeled into the underlying acquisition and/or distribution system and/or the absorbent core. One of the important qualities of a topsheet is the ability to reduce ponding of the fluids on the topsheets before the fluids are able to be absorbed by the absorbent article. Stated another way, one design criteria of topsheets is to reduce the amount of time the fluids spend on the topsheets prior to being absorbed by the absorbent article. If the fluids remain on the surfaces of the topsheets for too long of a period of time, wearers may not feel dry and discomfort may increase.

To solve the problem of wearer's skin feeling wet during, for example, a urination event, because of prolonged fluid residency on a topsheet, apertured topsheets have been used to allow for faster fluid penetration into the absorbent article. Although apertured topsheets have generally reduced fluid pendency on topsheets, topsheets can still be further improved by providing three-dimensional substrates that further reduce skin/fluid contact and/or skin/fluid contact time during, for example, a urination event.

### SUMMARY

The present disclosure is generally related, in part, to three-dimensional substrates that may be applied to topsheets of absorbent articles, form portions of, or all of, the topsheets, or form other portions of absorbent articles. The three-dimensional substrates may be liquid permeable substrates. The three-dimensional substrates of the present disclosure may reduce fluid/skin contact and/or fluid/skin contact time by providing first elements having a first z-directional height and at least second elements having a second z-directional height. The three-dimensional substrates may also have at least third elements having at least third z-directional heights. These substrates may also comprise apertures. The first z-directional height may generally be higher than the second z-directional height and the third z-directional height. In other instances, the first z-directional heights may be the same as, different than, or less than the second z-directional heights. The third z-directional height may be different than the second z-directional height, such as greater than or less than. Such a structure creates a substrate having a plurality of z-directional heights, such as two, three, or more z-directional heights. These three-dimensional substrates may allow fluids, during a urination event, for example, to be received onto the substrate and moved into the second elements having the second z-directional height (if lower than first z-directional height), the third elements having the third z-directional height (lower than first z-directional height) and/or into and through the apertures to at least reduce the amount of fluid in contact with the skin and/or to at least reduce the fluid/skin contact time. Stated another way, the first elements having the first z-directional height (if higher) may be in contact with the skin, while the fluids moves via gravity into the second elements having the second z-directional height, the third elements having the third z-directional height, and/or into and through the apertures. Upon information and belief, such three-dimensional structures reduce the amount of fluid on skin, give the wearer a drier, more comfortable feel, and/or reduce the pendency of fluid/skin contact. The first elements having the first z-directional height (if higher) essentially serve to provide a spacer between the skin and the fluids while the substrates are channeling the fluids into the acquisition and/or distribution system and/or the absorbent core.

### BRIEF DESCRIPTION OF THE DRAWINGS

The above-mentioned and other features and advantages of the present disclosure, and the manner of attaining them, will become more apparent and the disclosure itself will be better understood by reference to the following description of non-limiting forms of the disclosure taken in conjunction with the accompanying drawings, wherein:

FIG. 1 is a top view of an absorbent article, wearer-facing surface facing the viewer, with some layers partially removed in accordance with the present disclosure;

FIG. 2 is a cross-sectional view of the absorbent article taken about line 2-2 of FIG. 1 in accordance with the present disclosure;

FIG. 3 is a cross-sectional view of the absorbent article taken about line 2-2 of FIG. 2 where the absorbent article has been loaded with fluid in accordance with the present disclosure;

FIG. 4 is a top view of another absorbent article, wearer-facing surface facing the viewer, with some layers partially removed in accordance with the present disclosure;

FIG. 5 is a cross-sectional view of the absorbent article taken about line 5-5 of FIG. 4 in accordance with the present disclosure;

FIG. 6 is a top view of an absorbent core of the absorbent article of FIG. 4 with some layers partially removed in accordance the present disclosure;

FIG. 7 is a cross-sectional view of the absorbent core taken about line 7-7 of FIG. 6 in accordance with the present disclosure;

FIG. 8 is a cross-sectional view of the absorbent core taken about line 8-8 of FIG. 6 in accordance with the present disclosure;

FIG. 9 is a top view of an absorbent article, wearer-facing surface facing the viewer, that is a sanitary napkin with some of the layers cut away in accordance with the present disclosure;

FIG. 10 is a schematic illustration of a three-dimensional, liquid permeable substrate positioned on and/or joined to a topsheet for an absorbent article in accordance with the present disclosure;

FIG. 11 is another schematic illustration of a three-dimensional, liquid permeable substrate positioned on and/or joined to a topsheet for an absorbent article in accordance with the present disclosure;

FIG. 12 is another schematic illustration of a three-dimensional, liquid permeable substrate positioned on and/or joined to a topsheet for an absorbent article in accordance with the present disclosure;

FIG. 13 is a top view of an illustration of a first three-dimensional, liquid permeable substrate, wearer-facing surface facing the viewer, in accordance with the present disclosure;

FIG. 14 is a front view of a portion of the first liquid permeable substrate, wearer-facing surface facing the viewer, in accordance with the present disclosure;

FIG. 15 is a perspective view of the portion of the first liquid permeable substrate of FIG. 14 in accordance with the present disclosure;

FIG. 16 is another front view, taken from a different portion of the first liquid permeable substrate, wearer-facing surface facing the viewer, in accordance with the present disclosure;

FIG. 17 is a perspective view of the different portion of the first liquid permeable substrate of FIG. 16 in accordance with the present disclosure;

FIG. 18 is a rear view of a portion of the first liquid permeable substrate of FIG. 14, garment-facing surface facing the viewer, in accordance with the present disclosure;

FIG. 19 is a perspective view of the portion of the first liquid permeable substrate of FIG. 18 in accordance with the present disclosure;

FIG. 20 is another rear view, taken from the different portion of the first liquid permeable substrate of FIG. 16, garment-facing surface facing the viewer, in accordance with the present disclosure;

FIG. 21 is a perspective view of the different portion of the first liquid permeable substrate of FIG. 20 in accordance with the present disclosure;

FIG. 22 is a cross-sectional view of the first liquid permeable substrate in accordance with the present disclosure;

FIG. 23 is a top view of an illustration of a second three-dimensional, liquid permeable substrate, wearer-facing surface facing the viewer, in accordance with the present disclosure;

FIG. 24 is a front view of a portion of the second liquid permeable substrate, wearer-facing surface facing the viewer, in accordance with the present disclosure;

FIG. 25 is a perspective view of the portion of the second liquid permeable substrate of FIG. 24 in accordance with the present disclosure;

FIG. 26 is another front view, taken from a different portion of the second liquid permeable substrate, wearer-facing surface facing the viewer, in accordance with the present disclosure;

FIG. 27 is a perspective view of the different portion of the second liquid permeable substrate of FIG. 26 in accordance with the present disclosure;

FIG. 28 is a rear view of a portion of the second liquid permeable substrate of FIG. 24, garment-facing surface facing the viewer, in accordance with the present disclosure;

FIG. 29 is a perspective view of the portion of the second liquid permeable substrate of FIG. 28 in accordance with the present disclosure;

FIG. 30 is another rear view, taken from the different portion of the second liquid permeable substrate of FIG. 26, garment-facing surface facing the viewer, in accordance with the present disclosure;

FIG. 31 is a perspective view of the different portion of the second liquid permeable substrate of FIG. 30 in accordance with the present disclosure;

FIG. 32 is a cross-sectional view of the second liquid permeable substrate in accordance with the present disclosure;

FIG. 33 is a top view of an illustration of a third three-dimensional, liquid permeable substrate, wearer-facing surface facing the viewer, in accordance with the present disclosure;

FIG. 34 is a front view of a portion of the third liquid permeable substrate, wearer-facing surface facing the viewer, in accordance with the present disclosure;

FIG. 35 is a perspective view of the portion of the third liquid permeable substrate in accordance with the present disclosure;

FIG. 36 is another front view, taken from a different portion of the third liquid permeable substrate, wearer-facing surface facing the viewer, in accordance with the present disclosure;

FIG. 37 is a perspective view of the different portion of the third liquid permeable substrate of FIG. 36 in accordance with the present disclosure;

FIG. 38 is a rear view of a portion of the third liquid permeable substrate of FIG. 34, garment-facing surface facing the viewer, in accordance with the present disclosure;

FIG. 39 is a perspective view of the portion of the third liquid permeable substrate of FIG. 38 in accordance with the present disclosure;

FIG. 40 is another rear view, taken from the different portion of the third liquid permeable substrate of FIG. 36, garment-facing surface facing the viewer, in accordance with the present disclosure;

FIG. 41 is a perspective view of the different portion of the third liquid permeable substrate of FIG. 40 in accordance with the present disclosure;

FIG. 42 is a cross-sectional view of the third liquid permeable substrate in accordance with the present disclosure; and

FIG. 43 is a side view of a package of absorbent articles in accordance with the present disclosure. The outer surface is illustrated as transparent for purposes of clarity.

### DETAILED DESCRIPTION

Various non-limiting forms of the present disclosure will now be described to provide an overall understanding of the principles of the structure, function, manufacture, and use of the three-dimension substrates disclosed herein. One or more examples of these non-limiting embodiments are illustrated in the accompanying drawings. Those of ordinary skill in the art will understand that the three-dimensional substrates described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### INTRODUCTION

As used herein, the term “absorbent article” refers to disposable devices such as infant, child, or adult diapers, adult incontinence products, training pants, sanitary napkins, and the like which are placed against or in proximity to a body of a wearer to absorb and contain the various fluids (urine, menses, and/or runny BM) or bodily exudates (generally solid BM) discharged from the body. Typically, these absorbent articles comprise a topsheet, backsheet, an absorbent core, optionally an acquisition system and/or a distribution system (which may be comprised of one or several layers), and typically other components, with the absorbent core normally placed at least partially between the backsheet and the acquisition and/or distribution system or between the topsheet and the backsheet. The absorbent articles comprising three-dimensional, liquid permeable substrates of the present disclosure will be further illustrated in the below description and in the Figures in the form of one or more components of taped diaper. Nothing in this description should be, however, considered limiting the scope of the claims. As such the present disclosure applies to any suitable form of absorbent articles (e.g., diapers, training pants, adult incontinence products, sanitary napkins).

As used herein, the term “nonwoven web” means a manufactured sheet, web, or batt of directionally or randomly orientated fibers, bonded by friction, and/or cohesion, and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers may have diameters ranging from less than about 0.001 mm to more than about 0 2 mm and may come in several different forms such as short fibers (known as staple, or chopped), continuous single fibers (filaments or monofilaments), untwisted bundles of continuous filaments (tow), and twisted bundles of continuous filaments (yam). Nonwoven webs may be formed by many processes such as meltblowing, spunbonding, solvent spinning, electrospinning, carding, and airlaying. The basis weight of nonwoven webs is usually expressed in grams per square meter (g/m² or gsm).

As used herein, the terms “joined”, “bonded”, or “attached” encompasses configurations whereby an element is directly secured to another element by affixing the element directly to the other element, and configurations whereby an element is indirectly secured to another element by affixing the element to intermediate member(s) which in turn are affixed to the other element.

As used herein, the term “machine direction” or “MD” is the direction that is substantially parallel to the direction of travel of a substrate as it is made. The “cross direction” or “CD” is the direction substantially perpendicular to the MD and in the plane generally defined by the substrate.

As used herein, the term “hydrophilic”, refers to a material having a contact angle less than or equal to 90° according to The American Chemical Society Publication “Contact Angle, Wettability, and Adhesion,” edited by Robert F. Gould and copyrighted in 1964.

As used herein, the term “hydrophobic”, refers to a material or layer having a contact angle greater than or equal to 90° according to The American Chemical Society Publication “Contact Angle, Wettability, and Adhesion,” edited by Robert F. Gould and copyrighted in 1964.

### General Description of the Absorbent Article

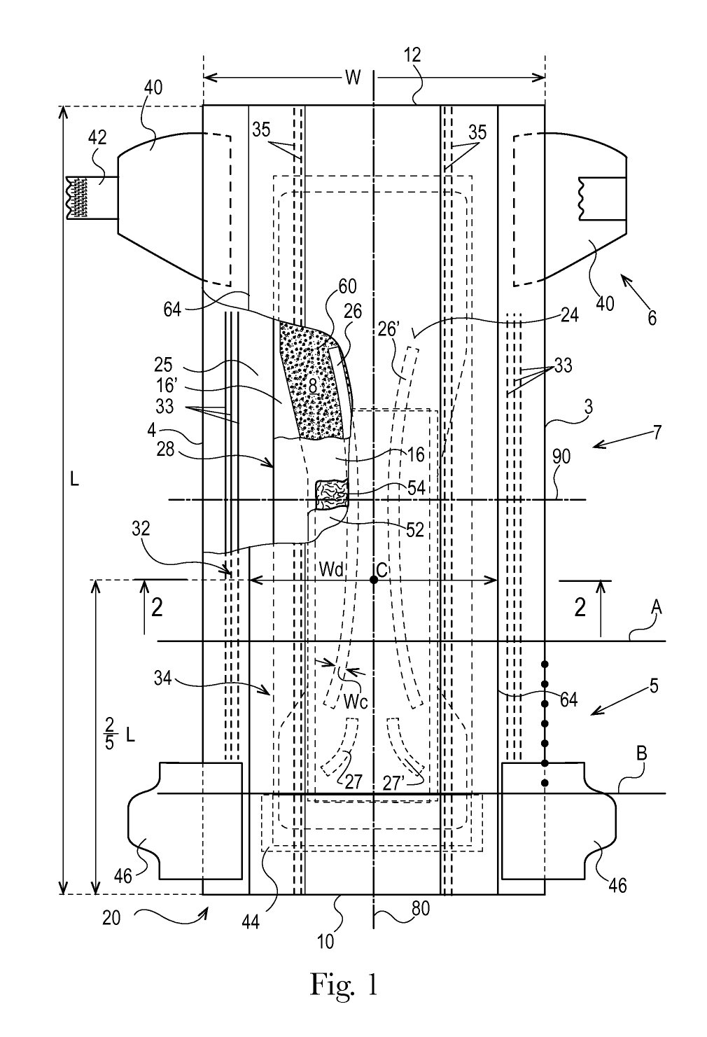

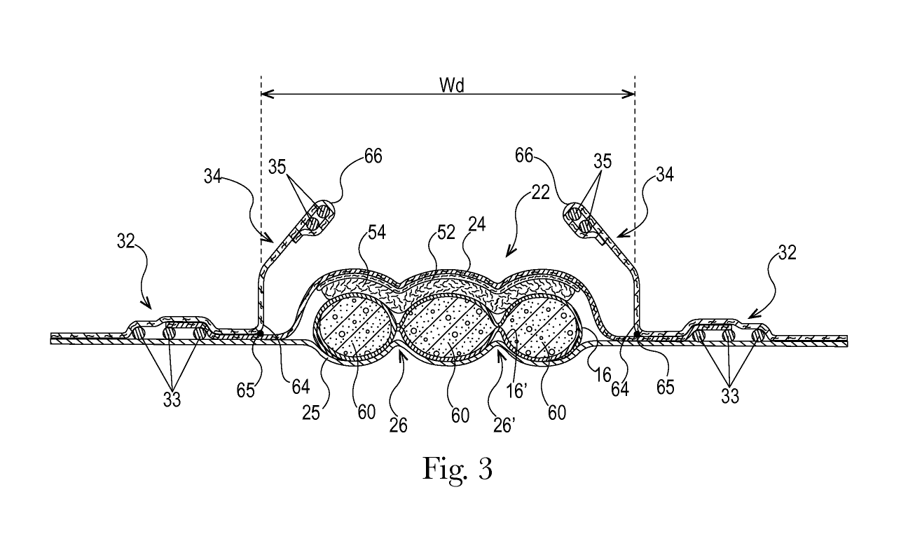

An example absorbent article in the form of a diaper 20 is represented in FIGS. 1-3. FIG. 1 is a plan view of the example diaper 20, in a flat-out state, with portions of the structure being cut-away to more clearly show the construction of the diaper 20. The wearer-facing surface of the diaper 20 of FIG. 1 is facing the viewer. This diaper 20 is shown for illustration purpose only as the three-dimensional substrates of the present disclosure may be used as one or more components of an absorbent article.

The absorbent article 20 may comprise a liquid permeable topsheet 24, a liquid impermeable backsheet 25, an absorbent core 28 positioned at least partially intermediate the topsheet 24 and the backsheet 25, and barrier leg cuffs 34. The absorbent article may also comprise an acquisition and/or distribution system (“ADS”) 50, which in the example represented comprises a distribution layer 54 and an acquisition layer 52, which will be further detailed below. The absorbent article may also comprise elasticized gasketing cuffs 32 comprising elastics 33 joined to a chassis of the absorbent article, typically via the topsheet and/or backsheet, and substantially planar with the chassis of the diaper.

The figures also show typical taped diaper components such as a fastening system comprising tabs 42 attached towards the rear edge of the article and cooperating with a landing zone 44 on the front of the absorbent article. The absorbent article may also comprise other typical elements, which are not represented, such as a rear elastic waist feature, a front elastic waist feature, transverse barrier cuff(s), and/or a lotion application, for example.

The absorbent article 20 comprises a front waist edge 10, a rear waist edge 12 longitudinally opposing the front waist edge 10, a first side edge 3, and a second side edge 4 laterally opposing the first side edge 3. The front waist edge 10 is the edge of the article which is intended to be placed towards the front of the user when worn, and the rear waist edge 12 is the opposite edge. The absorbent article may have a longitudinal axis 80 extending from the lateral midpoint of the front waist edge 10 to a lateral midpoint of the rear waist edge 12 of the article and dividing the article in two substantially symmetrical halves relative to the longitudinal axis 80, with the article placed flat and viewed from above as in FIG. 1. The absorbent article may also have a lateral axis 90 extending from the longitudinal midpoint of the first side edge 3 to the longitudinal midpoint of the second side edge 4. The length, L, of the article may be measured along the longitudinal axis 80 from the front waist edge 10 to the rear waist edge 12. The width, W, of the article may be measured along the lateral axis 90 from the first side edge 3 to the second side edge 4. The article may comprise a crotch point C defined herein as the point placed on the longitudinal axis at a distance of two fifth (⅖) of L starting from the front edge 10 of the article 20. The article may comprise a front waist region 5, a rear waist region 6, and a crotch region 7. The front waist region 5, the rear waist region 6, and the crotch region 7 each define ⅓ of the longitudinal length, L, of the absorbent article.

The topsheet 24, the backsheet 25, the absorbent core 28, and the other article components may be assembled in a variety of configurations, in particular by gluing or heat embossing, for example. Example absorbent article configurations are described generally in U.S. Pat. No. 3,860,003, U.S. Pat. No. 5,221,274, U.S. Pat. No. 5,554,145, U.S. Pat. No. 5,569,234, U.S. Pat. No. 5,580,411, and U.S. Pat. No. 6,004,306.

The absorbent core 28 may comprise an absorbent material comprising at least 80% by weight, at least 90% by weight, at least 95% by weight, or at least 99% by weight of superabsorbent polymers and a core wrap enclosing the superabsorbent polymers. The core wrap may typically comprise two materials, substrates, or nonwoven materials 16 and 16′ for the top side and bottom side of the core. The core may comprises one or more channels, represented in FIG. 1 as the four channels 26, 26′ and 27, 27′. The channels 26, 26′, 27, and 27′ are optional features. Instead, the core may not have any channels or may have any number of channels.

These and other components of the example absorbent article will now be discussed in more details.

### Topsheet

In the present disclosure, the topsheet (the portion of the absorbent article that contacts the wearer's skin and receives the fluids) may be formed of a portion of, or all of, one or more of the three-dimensional substrates described herein and/or have one or more three-dimensional substrates positioned thereon and/or joined thereto, so that the three-dimensional substrate(s) contact(s) the wearer's skin. Other portions of the topsheet (other than the three-dimensional substrates) may also contact the wearer's skin. A typical topsheet is described below, although it will be understood that this topsheet 24, or portions thereof, may be replaced by the three-dimensional substrates described herein. Alternatively, the three-dimensional substrates may be positioned as a strip or a patch on top of the typical topsheet 24, as is described herein.

The topsheet 24 may be the part of the absorbent article that is in contact with the wearer's skin. The topsheet 24 may be joined to the backsheet 25, the core 28 and/or any other layers as is known to those of skill in the art. Usually, the topsheet 24 and the backsheet 25 are joined directly to each other in some locations (e.g., on or close to the periphery of the absorbent article) and are indirectly joined together in other locations by directly joining them to one or more other elements of the article 20.

The topsheet 24 may be compliant, soft-feeling, and non-irritating to the wearer's skin. Further, a portion of, or all of, the topsheet 24 may be liquid permeable, permitting liquids to readily penetrate through its thickness. A suitable topsheet may be manufactured from a wide range of materials, such as porous foams, reticulated foams, apertured plastic films, or woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. If the topsheet 24 includes fibers, the fibers may be spunbond, carded, wet-laid, meltblown, hydroentangled, or otherwise processed as is known in the art. A suitable topsheet comprising a web of spunbond polypropylene (topically treated with a hydrophilic surfactant) is manufactured by Polymer Group, Inc., of Charlotte, N.C., under the designation P-10.

Any portion of the topsheet 24 may be coated with a lotion and/or a skin care composition as is generally disclosed in the art. The topsheet 24 may also comprise or be treated with antibacterial agents, some examples of which are disclosed in PCT Publication WO95/24173. Further, the topsheet 24, the backsheet 25 or any portion of the topsheet or backsheet may be embossed and/or matte finished to provide a more cloth like appearance.

The topsheet 24 may comprise one or more apertures to ease penetration of fluids therethrough. The size of at least the primary apertures is important in achieving the desired fluid encapsulation performance. If the primary apertures are too small, the fluids may not pass through the apertures, either due to poor alignment of the fluid source and the aperture location or due to runny fecal masses, for example, having a diameter greater than the apertures. If the apertures are too large, the area of skin that may be contaminated by “rewet” from the article is increased. Typically, the total area of the apertures at the surface of a diaper may have an area of between about 10 cm² and about 50 cm² or between about 15 cm² and 35 cm². Examples of apertured topsheets are disclosed in U.S. Pat. No. 6,632,504, assigned to BBA NONWOVENS SIMPSONVILLE. Typical diaper topsheets have a basis weight of from about 10 gsm to about 50 gsm or from about 12 gsm to about 30 gsm, but other basis weights are within the scope of the present disclosure.

### Backsheet

The backsheet 25 is generally that portion of the absorbent article 20 positioned adjacent the garment-facing surface of the absorbent core 28 and which prevents, or at least inhibits, the fluids and bodily exudates absorbed and contained therein from soiling articles such as bedsheets and undergarments. The backsheet 25 is typically impermeable, or at least substantially impermeable, to fluids (e.g., urine). The backsheet may, for example, be or comprise a thin plastic film such as a thermoplastic film having a thickness of about 0.012 mm to about 0.051 mm. Example backsheet films include those manufactured by Tredegar Corporation, based in Richmond, Va., and sold under the trade name CPC2 film. Other suitable backsheet materials may include breathable materials which permit vapors to escape from the absorbent article 20 while still preventing, or at least inhibiting, fluids from passing through the backsheet 25. Example breathable materials may include materials such as woven webs, nonwoven webs, composite materials such as film-coated nonwoven webs, microporous films such as manufactured by Mitsui Toatsu Co., of Japan under the designation ESPOIR NO and by Tredegar Corporation of Richmond, Va., and sold under the designation EXAIRE, and monolithic films such as manufactured by Clopay Corporation, Cincinnati, Ohio under the name HYTREL blend P18-3097.

The backsheet 25 may be joined to the topsheet 24, the absorbent core 28, and/or any other element of the absorbent article 20 by any attachment methods known to those of skill in the art. Suitable attachment methods are described above with respect to methods for joining the topsheet 24 to other elements of the article 20.

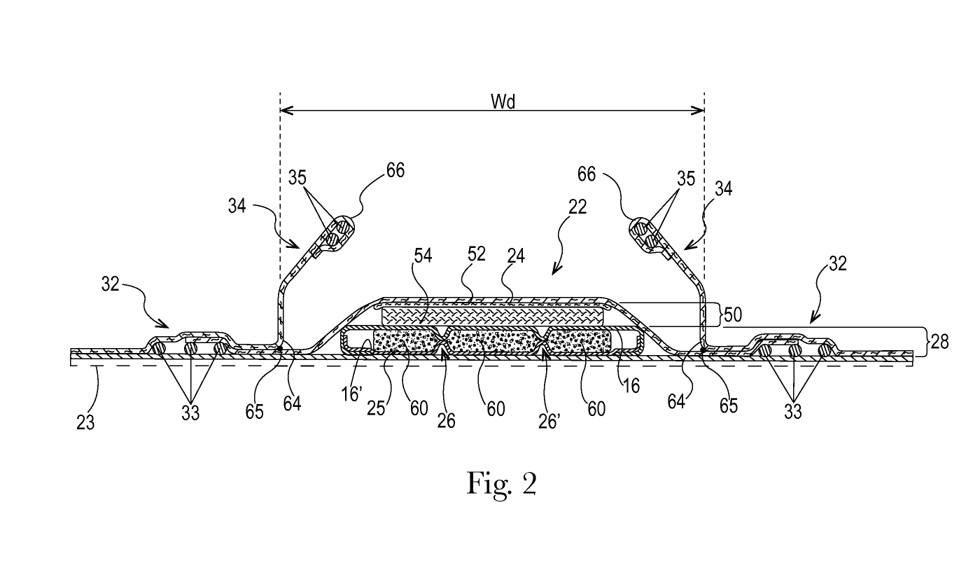

An outer cover 23 may cover at least a portion of, or all of, the backsheet 25 to form a soft garment-facing surface of the absorbent article. The outer cover 23 may be formed of one or more nonwoven materials. The outer cover 23 is illustrated in dash in FIG. 2, as an example. The outer cover 23 may be joined to at least a portion of the backsheet 25 through mechanical bonding, adhesive bonding, or other suitable methods of attachment.

### Absorbent Core

As used herein, the term “absorbent core” refers to the component of the absorbent article having the most absorbent capacity and comprising an absorbent material and a core wrap or core bag enclosing the absorbent material. The term “absorbent core” does not include the acquisition and/or distribution system or any other components of the article which are not either integral part of the core wrap or core bag or placed within the core wrap or core bag. The absorbent core may comprise, consist essentially of, or consist of, a core wrap, an absorbent material (e.g., superabsorbent polymers) as discussed, and glue.

The absorbent core 28 may comprise an absorbent material with a high amount of superabsorbent polymers (herein abbreviated as “SAP”) enclosed within the core wrap. The SAP content may represent 70%-100% or at least 70%, 75%, 80%, 85%, 90%, 95%, 99%, or 100%, by weight of the absorbent material, contained in the core wrap. The core wrap is not considered as absorbent material for the purpose of assessing the percentage of SAP in the absorbent core. The core may also contain airfelt or cellulosic fibers with or without SAP.

By “absorbent material” it is meant a material which has some absorbency property or liquid retaining properties, such as SAP, cellulosic fibers as well as synthetic fibers. Typically, glues used in making absorbent cores have no or little absorbency properties and are not considered as absorbent material. The SAP content may be higher than 80%, for example at least 85%, at least 90%, at least 95%, at least 99%, and even up to and including 100% of the weight of the absorbent material contained within the core wrap. This provides a relatively thin core compared to a conventional core typically comprising between 40-60% SAP and high content of cellulose fibers. The conventional cores are also within the scope of the present disclosure. The absorbent material may in particular comprises less than 15% weight percent or less than 10% weight percent of natural, cellulosic, or synthetic fibers, less than 5% weight percent, less than 3% weight percent, less than 2% weight percent, less than 1% weight percent, or may even be substantially free of natural, cellulosic, and/or synthetic fibers.

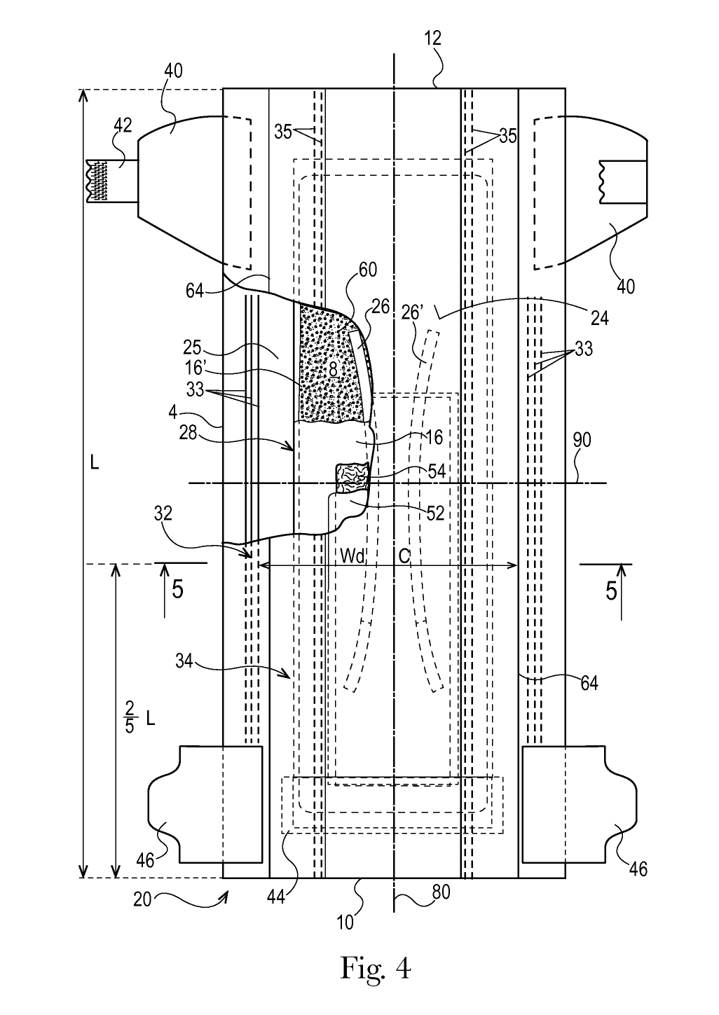

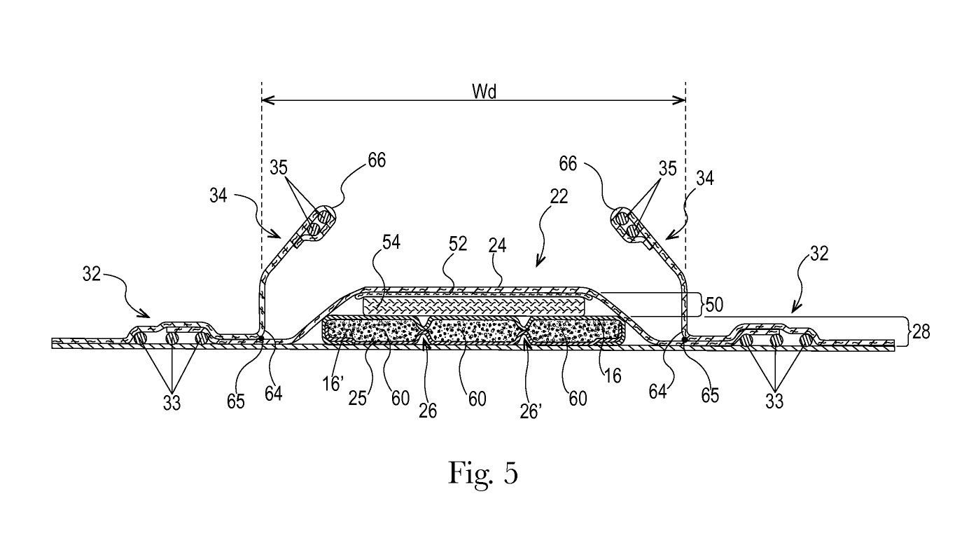

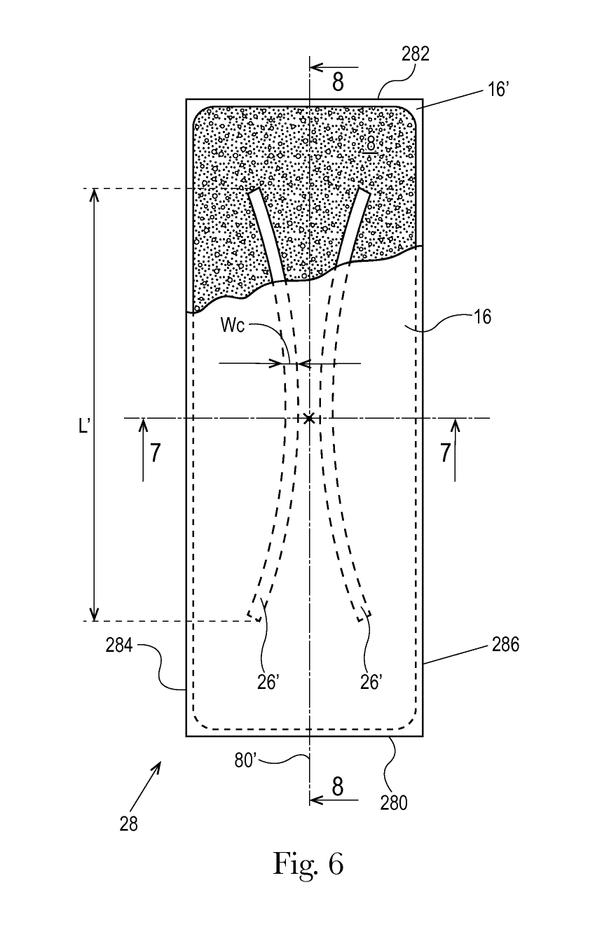

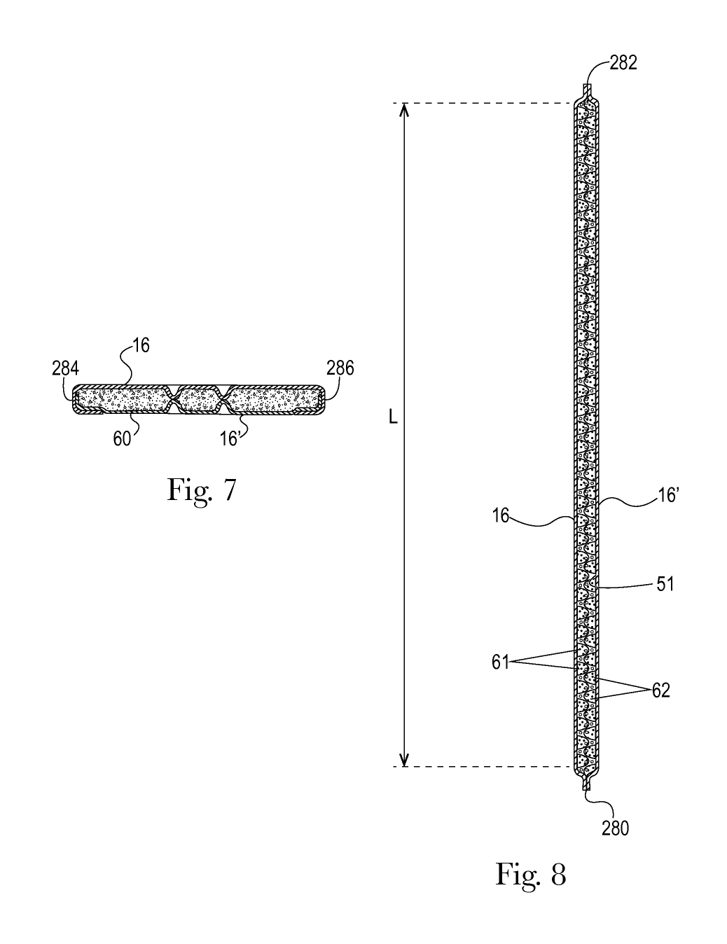

The example absorbent core 28 of the absorbent article 20 of FIGS. 4-5 is shown in isolation in FIGS. 6-8. The absorbent core 28 may comprises a front side 280, a rear side 282, and two longitudinal sides 284, 286 joining the front side 280 and the rear side 282. The absorbent core 28 may also comprise a generally planar top side and a generally planar bottom side. The front side 280 of the core is the side of the core intended to be placed towards the front waist edge 10 of the absorbent article. The core 28 may have a longitudinal axis 80′ corresponding substantially to the longitudinal axis 80 of the absorbent article 20, as seen from the top in a planar view as in FIG. 1. The absorbent material may be distributed in higher amount towards the front side 280 than towards the rear side 282 as more absorbency may be required at the front in particular absorbent articles. The front and rear sides 280 and 282 of the core may be shorter than the longitudinal sides 284 and 286 of the core. The core wrap may be formed by two nonwoven materials, substrates, laminates, or other materials, 16, 16′ which may be at least partially sealed along the sides 284, 286 of the absorbent core 28. The core wrap may be at least partially sealed along its front side 280, rear side 282, and two longitudinal sides 284, 286 so that substantially no absorbent material leaks out of the absorbent core wrap. The first material, substrate, or nonwoven 16 may at least partially surround the second material, substrate, or nonwoven 16′ to form the core wrap, as illustrated in FIG. 7. The first material 16 may surround a portion of the second material 16′ proximate to the first and second side edges 284 and 286.

The absorbent core may comprise adhesive, for example, to help immobilizing the SAP within the core wrap and/or to ensure integrity of the core wrap, in particular when the core wrap is made of two or more substrates. The adhesive may be a hot melt adhesive, supplied, by H. B. Fuller, for example. The core wrap may extend to a larger area than strictly needed for containing the absorbent material within.

Cores comprising relatively high amount of SAP with various core designs are disclosed in U.S. Pat. No. 5,599,335 (Goldman), EP 1,447,066 (Busam), WO 95/11652 (Tanzer), U.S. Pat. Publ. No. 2008/0312622A1 (Hundorf), and WO 2012/052172 (Van Malderen).

The absorbent material may be a continuous layer present within the core wrap. Alternatively, the absorbent material may be comprised of individual pockets or stripes of absorbent material enclosed within the core wrap. In the first case, the absorbent material may be, for example, obtained by the application of a single continuous layer of absorbent material. The continuous layer of absorbent material, in particular of SAP, may also be obtained by combining two absorbent layers having discontinuous absorbent material application patterns, wherein the resulting layer is substantially continuously distributed across the absorbent particulate polymer material area, as disclosed in U.S. Pat. Appl. Pub. No. 2008/0312622A1 (Hundorf), for example. The absorbent core 28 may comprise a first absorbent layer and a second absorbent layer. The first absorbent layer may comprise the first material 16 and a first layer 61 of absorbent material, which may be 100% or less of SAP. The second absorbent layer may comprise the second material 16′ and a second layer 62 of absorbent material, which may also be 100% or less of SAP. The absorbent core 28 may also comprise a fibrous thermoplastic adhesive material 51 at least partially bonding each layer of absorbent material 61, 62 to its respective material 16 or 16′. This is illustrated in FIGS. 7-8, as an example, where the first and second SAP layers have been applied as transversal stripes or “land areas” having the same width as the desired absorbent material deposition area on their respective substrate before being combined. The stripes may comprise different amounts of absorbent material (SAP) to provide a profiled basis weight along the longitudinal axis of the core 80. The first material 16 and the second material 16′ may form the core wrap.

The fibrous thermoplastic adhesive material 51 may be at least partially in contact with the absorbent material 61, 62 in the land areas and at least partially in contact with the materials 16 and 16′ in the junction areas. This imparts an essentially three-dimensional structure to the fibrous layer of thermoplastic adhesive material 51, which in itself is essentially a two-dimensional structure of relatively small thickness, as compared to the dimension in length and width directions. Thereby, the fibrous thermoplastic adhesive material may provide cavities to cover the absorbent material in the land areas, and thereby immobilizes this absorbent material, which may be 100% or less of SAP.

The thermoplastic adhesive used for the fibrous layer may have elastomeric properties, such that the web formed by the fibers on the SAP layer is able to be stretched as the SAP swell. Elastomeric, hot-melt adhesives of these types are described in more detail in U.S. Pat. No. 4,731,066 issued to Korpman on Mar. 15, 1988. The thermoplastic adhesive material may be applied as fibers.

### Superabsorbent Polymer (SAP)

“Superabsorbent polymers” (“SAP”), as used herein, refer to absorbent materials which are cross-linked polymeric materials that can absorb at least 10 times their weight of an aqueous 0.9% saline solution as measured using the Centrifuge Retention Capacity (CRC) test (EDANA method WSP 241.2-05E). The SAP used may have a CRC value of more than 20 g/g, more than 24 g/g, from 20 to 50 g/g, from 20 to 40 g/g, or from 24 to 30 g/g, specifically reciting all 0.1 g/g increments within the above-specified ranges and any ranges created therein or thereby. The SAP useful with the present disclosure may include a variety of water-insoluble, but water-swellable polymers capable of absorbing large quantities of fluids.

The superabsorbent polymer may be in particulate form so as to be flowable in the dry state. Particulate absorbent polymer materials may be made of poly(meth)acrylic acid polymers. However, starch-based particulate absorbent polymer material may also be used, as well as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

The SAP may be of numerous shapes. The term “particles” refers to granules, fibers, flakes, spheres, powders, platelets and other shapes and forms known to persons skilled in the art of superabsorbent polymer particles. The SAP particles may be in the shape of fibers, i.e., elongated, acicular superabsorbent polymer particles. The fibers may also be in the form of a long filament that may be woven. SAP may be spherical-like particles. The absorbent core may comprise one or more types of SAP.

For most absorbent articles, liquid discharges from a wearer occur predominately in the front half of the absorbent article, in particular for a diaper. The front half of the article (as defined by the region between the front edge and a transversal line placed at a distance of half L from the front waist edge 10 or rear waist edge 12 may therefore may comprise most of the absorbent capacity of the core. Thus, at least 60% of the SAP, or at least 65%, 70%, 75%, 80%, or 85% of the SAP may be present in the front half of the absorbent article, while the remaining SAP may be disposed in the rear half of the absorbent article. Alternatively, the SAP distribution may be uniform through the core or may have other suitable distributions.

The total amount of SAP present in the absorbent core may also vary according to expected user. Diapers for newborns may require less SAP than infant, child, or adult incontinence diapers. The amount of SAP in the core may be about 5 to 60 g or from 5 to 50 g, specifically reciting all 0.1 increments within the specified ranges and any ranged formed therein or thereby. The average SAP basis weight within the (or “at least one”, if several are present) deposition area 8 of the SAP may be at least 50, 100, 200, 300, 400, 500 or more g/m². The areas of the channels (e.g., 26, 26′, 27, 27′) present in the absorbent material deposition area 8 are deduced from the absorbent material deposition area to calculate this average basis weight.

### Core Wrap

The core wrap may be made of a single substrate, material, or nonwoven folded around the absorbent material, or may comprise two (or more) substrates, materials, or nonwovens which are attached to another. Typical attachments are the so-called C-wrap and/or sandwich wrap. In a C-wrap, as illustrated, for example, in FIGS. 2 and 7, the longitudinal and/or transversal edges of one of the substrates are folded over the other substrate to form flaps. These flaps are then bonded to the external surface of the other substrate, typically by gluing.

The core wrap may be formed by any materials suitable for receiving and containing the absorbent material. Typical substrate materials used in the production of conventional cores may be used, in particular paper, tissues, films, wovens or nonwovens, or laminates or composites of any of these.

The substrates may also be air-permeable (in addition to being liquid or fluid permeable). Films useful herein may therefore comprise micro-pores.

The core wrap may be at least partially sealed along all the sides of the absorbent core so that substantially no absorbent material leaks out of the core. By “substantially no absorbent material” it is meant that less than 5%, less than 2%, less than 1%, or about 0% by weight of absorbent material escape the core wrap. The term “seal” is to be understood in a broad sense. The seal does not need to be continuous along the whole periphery of the core wrap but may be discontinuous along part or the whole of it, such as formed by a series of seal points spaced on a line. A seal may be formed by gluing and/or thermal bonding.

If the core wrap is formed by two substrates 16, 16′, four seals may be used to enclose the absorbent material 60 within the core wrap. For example, a first substrate 16 may be placed on one side of the core (the top side as represented in the Figures) and extend around the core's longitudinal edges to at least partially wrap the opposed bottom side of the core. The second substrate 16′ may be present between the wrapped flaps of the first substrate 16 and the absorbent material 60. The flaps of the first substrate 16 may be glued to the second substrate 16′ to provide a strong seal. This so called C-wrap construction may provide benefits such as improved resistance to bursting in a wet loaded state compared to a sandwich seal. The front side and rear side of the core wrap may then also be sealed by gluing the first substrate and second substrate to another to provide complete encapsulation of the absorbent material across the whole of the periphery of the core. For the front side and rear side of the core, the first and second substrates may extend and may be joined together in a substantially planar direction, forming for these edges a so-called sandwich construction. In the so-called sandwich construction, the first and second substrates may also extend outwardly on all sides of the core and be sealed flat, or substantially flat, along the whole or parts of the periphery of the core typically by gluing and/or heat/pressure bonding. In an example, neither the first nor the second substrates need to be shaped, so that they may be rectangularly cut for ease of production but other shapes are within the scope of the present disclosure.

The core wrap may also be formed by a single substrate which may enclose as in a parcel wrap the absorbent material and be sealed along the front side and rear side of the core and one longitudinal seal.

### SAP Deposition Area

The absorbent material deposition area 8 may be defined by the periphery of the layer formed by the absorbent material 60 within the core wrap, as seen from the top side of the absorbent core. The absorbent material deposition area 8 may have various shapes, in particular, a so-called “dog bone” or “hour-glass” shape, which shows a tapering along its width towards the middle or “crotch” region of the core. In this way, the absorbent material deposition area 8 may have a relatively narrow width in an area of the core intended to be placed in the crotch region of the absorbent article, as illustrated in FIG. 1. This may provide better wearing comfort. The absorbent material deposition area 8 may also be generally rectangular, for example as shown in FIGS. 4-6, but other deposition areas, such as a rectangular, “T,” “Y,” “sand-hour,” or “dog-bone” shapes are also within the scope of the present disclosure. The absorbent material may be deposited using any suitable techniques, which may allow relatively precise deposition of SAP at relatively high speed.

### Channels

The absorbent material deposition area 8 may comprise at least one channel 26, which is at least partially oriented in the longitudinal direction of the article 80 (i.e., has a longitudinal vector component). Other channels may be at least partially oriented in the lateral direction (i.e., has a lateral vector component) or in any other direction. In the following, the plural form “channels” will be used to mean “at least one channel”. The channels may have a length L′ projected on the longitudinal axis 80 of the article that is at least 10% of the length L of the article. The channels may be formed in various ways. For example, the channels may be formed by zones within the absorbent material deposition area 8 which may be substantially free of, or free of, absorbent material, in particular SAP. In addition or alternatively, the channel(s) may also be formed by continuously or discontinuously bonding the top side of the core wrap to the bottom side of the core wrap through the absorbent material deposition area 8. The channels may be continuous but it is also envisioned that the channels may be intermittent. The acquisition-distribution system or layer 50, or another layer of the article, may also comprise channels, which may or not correspond to the channels of the absorbent core.

In some instances, the channels may be present at least at the same longitudinal level as the crotch point C or the lateral axis 60 in the absorbent article, as represented in FIG. 1 with the two longitudinally extending channels 26, 26′. The channels may also extend from the crotch region 7 or may be present in the front waist region 5 and/or in the rear waist region 6 of the article.

The absorbent core 28 may also comprise more than two channels, for example, at least 3, at least 4, at least 5, or at least 6 or more. Shorter channels may also be present, for example in the rear waist region 6 or the front waist region 5 of the core as represented by the pair of channels 27, 27′ in FIG. 1 towards the front of the article. The channels may comprise one or more pairs of channels symmetrically arranged, or otherwise arranged relative to the longitudinal axis 80.

The channels may be particularly useful in the absorbent core when the absorbent material deposition area is rectangular, as the channels may improve the flexibility of the core to an extent that there is less advantage in using a non-rectangular (shaped) core. Of course channels may also be present in a layer of SAP having a shaped deposition area.

The channels may be completely oriented longitudinally and parallel to the longitudinal axis or completely oriented transversely and parallel to the lateral axis, but also may have at least portions that are curved.

In order to reduce the risk of fluid leakages, the longitudinal main channels may not extend up to any of the edges of the absorbent material deposition area 8, and may therefore be fully encompassed within the absorbent material deposition area 8 of the core. The smallest distance between a channel and the closest edge of the absorbent material deposition area 8 may be at least 5 mm.

The channels may have a width We along at least part of their length which is at least 2 mm, at least 3 mm, at least 4 mm, up to for example 20 mm, 16 mm, or 12 mm, for example. The width of the channel(s) may be constant through substantially the whole length of the channel or may vary along its length. When the channels are formed by absorbent material-free zone within the absorbent material deposition area 8, the width of the channels is considered to be the width of the material free zone, disregarding the possible presence of the core wrap within the channels. If the channels are not formed by absorbent material free zones, for example mainly though bonding of the core wrap through the absorbent material zone, the width of the channels is the width of this bonding.

At least some or all of the channels may be permanent channels, meaning their integrity is at least partially maintained both in the dry state and in the wet state. Permanent channels may be obtained by provision of one or more adhesive materials, for example, the fibrous layer of adhesive material or construction glue that helps adhere a substrate with an absorbent material within the walls of the channel. Permanent channels may also be formed by bonding the upper side and lower side of the core wrap (e.g., the first substrate 16 and the second substrate 16′) and/or the topsheet 24 to the backsheet 25 together through the channels. Typically, an adhesive may be used to bond both sides of the core wrap or the topsheet and the backsheet through the channels, but it is possible to bond via other known processes, such as pressure bonding, ultrasonic bonding, heat bonding, or combination thereof. The core wrap or the topsheet 24 and the backsheet 25 may be continuously bonded or intermittently bonded along the channels. The channels may advantageously remain or become visible at least through the topsheet and/or backsheet when the absorbent article is fully loaded with a fluid. This may be obtained by making the channels substantially free of SAP, so they will not swell, and sufficiently large so that they will not close when wet. Furthermore, bonding the core wrap to itself or the topsheet to the backsheet through the channels may be advantageous.

### Barrier Leg Cuffs

The absorbent article may comprise a pair of barrier leg cuffs 34. Each barrier leg cuff may be formed by a piece of material which is bonded to the article so it may extend upwards from a wearer-facing surface of the absorbent article and provide improved containment of fluids and other body exudates approximately at the junction of the torso and legs of the wearer. The barrier leg cuffs are delimited by a proximal edge 64 joined directly or indirectly to the topsheet 24 and/or the backsheet 25 and a free terminal edge 66, which is intended to contact and form a seal with the wearer's skin. The barrier leg cuffs 34 extend at least partially between the front waist edge 10 and the rear waist edge 12 of the absorbent article on opposite sides of the longitudinal axis 80 and are at least present at the level of the crotch point (C) or crotch region. The barrier leg cuffs may be joined at the proximal edge 64 with the chassis of the article by a bond 65 which may be made by gluing, fusion bonding, or a combination of other suitable bonding processes. The bond 65 at the proximal edge 64 may be continuous or intermittent. The bond 65 closest to the raised section of the leg cuffs delimits the proximal edge 64 of the standing up section of the leg cuffs.

The barrier leg cuffs may be integral with the topsheet 24 or the backsheet 25 or may be a separate material joined to the article's chassis. Each barrier leg cuff 34 may comprise one, two or more elastic strings 35 close to the free terminal edge 66 to provide a better seal.

In addition to the barrier leg cuffs 34, the article may comprise gasketing cuffs 32, which are joined to the chassis of the absorbent article, in particular to the topsheet 24 and/or the backsheet 25 and are placed externally relative to the barrier leg cuffs. The gasketing cuffs 32 may provide a better seal around the thighs of the wearer. Each gasketing leg cuff may comprise one or more elastic strings or elastic elements 33 in the chassis of the absorbent article between the topsheet 24 and backsheet 25 in the area of the leg openings. All, or a portion of, the barrier leg cuffs and/or gasketing cuffs may be treated with a lotion or another skin care composition.

### Acquisition-Distribution System

The absorbent articles of the present disclosure may comprise an acquisition-distribution layer or system 50 (“ADS”). One function of the ADS is to quickly acquire one or more of the fluids and distribute them to the absorbent core in an efficient manner. The ADS may comprise one, two or more layers, which may form a unitary layer or may remain as discrete layers which may be attached to each other. In an example, the ADS may comprise two layers: a distribution layer 54 and an acquisition layer 52 disposed between the absorbent core and the topsheet, but the present disclosure is not so limited.

The ADS may comprise SAP as this may slow the acquisition and distribution of the fluids. Suitable ADS are described in WO 2000/59430 (Daley), WO 95/10996 (Richards), U.S. Pat. No. 5,700,254 (McDowall), and WO 02/067809 (Graef), for example.

In one example, the ADS may not be provided, or only one layer of the ADS may be provided, such as the distribution layer only or the acquisition layer only. When one of the three-dimensional, liquid permeable substrates of the present disclosure is used as a portion of, or all of, a topsheet, or positioned on a topsheet, dryness performance of the liquid permeable substrates may be improved if only one or no layers of the ADS are present. This is owing to the fact that fluids (e.g., urine) are easily able to wick through the liquid permeable substrates directly into the absorbent core 28 and/or into a one layer ADS.

### Distribution Layer

The distribution layer of the ADS may comprise at least 50% by weight of cross-linked cellulose fibers. The cross-linked cellulosic fibers may be crimped, twisted, or curled, or a combination thereof including crimped, twisted, and curled. This type of material is disclosed in U.S. Pat. Publ. No. 2008/0312622 A1 (Hundorf). The cross-linked cellulosic fibers provide higher resilience and therefore higher resistance to the first absorbent layer against the compression in the product packaging or in use conditions, e.g., under wearer weight. This may provide the core with a higher void volume, permeability, and liquid absorption, and hence reduced leakage and improved dryness.

The distribution layer comprising the cross-linked cellulose fibers of the present disclosure may comprise other fibers, but this layer may advantageously comprise at least 50%, or 60%, or 70%, or 80%, or 90%, or even up to 100%, by weight of the layer, of cross-linked cellulose fibers (including the cross-linking agents). Examples of such mixed layer of cross-linked cellulose fibers may comprise about 70% by weight of chemically cross-linked cellulose fibers, about 10% by weight polyester (PET) fibers, and about 20% by weight untreated pulp fibers. In another example, the layer of cross-linked cellulose fibers may comprise about 70% by weight chemically cross-linked cellulose fibers, about 20% by weight lyocell fibers, and about 10% by weight PET fibers. In still another example, the layer may comprise about 68% by weight chemically cross-linked cellulose fibers, about 16% by weight untreated pulp fibers, and about 16% by weight PET fibers. In yet another example, the layer of cross-linked cellulose fibers may comprise from about 90 to about 100% by weight chemically cross-linked cellulose fibers.

### Acquisition Layer

The ADS 50 may comprise an acquisition layer 52. The acquisition layer may be disposed between the distribution layer 54 and the topsheet 24. The acquisition layer 52 may be or may comprise a nonwoven material, such as a hydrophilic SMS or SMMS material, comprising a spunbonded, a melt-blown and a further spunbonded layer or alternatively a carded staple fiber chemical-bonded nonwoven. The nonwoven material may be latex bonded.

A further acquisition layer may be used in addition to a first acquisition layer described above. For example, a tissue layer may be placed between the first acquisition layer and the distribution layer. The tissue may have enhanced capillarity distribution properties compared to the acquisition layer described above.

### Fastening System

The absorbent article may include a fastening system. The fastening system may be used to provide lateral tensions about the circumference of the absorbent article to hold the absorbent article on the wearer as is typical for taped diapers. This fastening system may not be necessary for training pant articles since the waist region of these articles is already bonded. The fastening system may comprise a fastener such as tape tabs, hook and loop fastening components, interlocking fasteners such as tabs & slots, buckles, buttons, snaps, and/or hermaphroditic fastening components, although any other suitable fastening mechanisms are also within the scope of the present disclosure. A landing zone 44 is normally provided on the garment-facing surface of the front waist region 5 for the fastener to be releasably attached thereto.

### Front and Rear Ears

The absorbent article may comprise front ears 46 and rear ears 40. The ears may be an integral part of the chassis, such as formed from the topsheet 24 and/or backsheet 26 as side panels. Alternatively, as represented on FIG. 1, the ears may be separate elements attached by gluing, heat embossing, and/or pressure bonding. The rear ears 40 may be stretchable to facilitate the attachment of the tabs 42 to the landing zone 44 and maintain the taped diapers in place around the wearer's waist. The rear ears 40 may also be elastic or extensible to provide a more comfortable and contouring fit by initially conformably fitting the absorbent article to the wearer and sustaining this fit throughout the time of wear well past when absorbent article has been loaded with fluids or other bodily exudates since the elasticized ears allow the sides of the absorbent article to expand and contract.

### Elastic Waist Feature

The absorbent article 20 may also comprise at least one elastic waist feature (not represented) that helps to provide improved fit and containment. The elastic waist feature is generally intended to elastically expand and contract to dynamically fit the wearer's waist. The elastic waist feature may extend at least longitudinally outwardly from at least one waist edge of the absorbent core 28 and generally forms at least a portion of the end edge of the absorbent article. Disposable diapers may be constructed so as to have two elastic waist features, one positioned in the front waist region and one positioned in the rear waist region.

### Relations Between the Layers

Typically, adjacent layers and components may be joined together using conventional bonding methods, such as adhesive coating via slot coating or spraying on the whole or part of the surface of the layer, thermo-bonding, pressure bonding, or combinations thereof. This bonding is not represented in the Figures (except for the bonding between the raised element of the leg cuffs 65 with the topsheet 24) for clarity and readability, but bonding between the layers of the article should be considered to be present unless specifically excluded. Adhesives may be used to improve the adhesion of the different layers between the backsheet 25 and the core wrap. The glue may be any suitable hotmelt glue known in the art.

### Sanitary Napkin

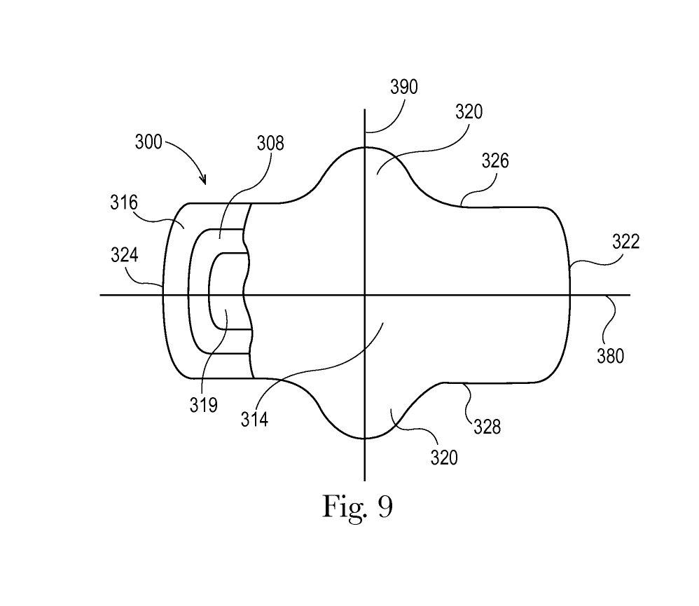

The three-dimensional substrates of the present disclosure may form a portion of a topsheet, form the topsheet, form a portion of, or all of a secondary topsheet, or be positioned on or joined to at least a portion of the topsheet of a sanitary napkin. Referring to FIG. 9, the absorbent article may comprise a sanitary napkin 300. The sanitary napkin 300 may comprise a liquid permeable topsheet 314, a liquid impermeable, or substantially liquid impermeable, backsheet 316, and an absorbent core 308. The absorbent core 308 may have any or all of the features described herein with respect to the absorbent cores 28 and, in some forms, may have a secondary topsheet instead of the acquisition-distribution system disclosed above. The sanitary napkin 300 may also comprise wings 320 extending outwardly with respect to a longitudinal axis 380 of the sanitary napkin 300. The sanitary napkin 300 may also comprise a lateral axis 390. The wings 320 may be joined to the topsheet 314, the backsheet 316, and/or the absorbent core 308. The sanitary napkin 300 may also comprise a front edge 322, a rear edge 324 longitudinally opposing the front edge 322, a first side edge 326, and a second side edge 328 longitudinally opposing the first side edge 326. The longitudinal axis 380 may extend from a midpoint of the front edge 322 to a midpoint of the rear edge 324. The lateral axis 390 may extend from a midpoint of the first side edge 326 to a midpoint of the second side edge 328. The sanitary napkin 300 may also be provided with additional features commonly found in sanitary napkins as is generally known in the art, such as a secondary topsheet 319, for example.

### Three-Dimensional Substrates

The three-dimensional, liquid permeable substrates of the present disclosure may comprise substrates that have first elements that have a first z-directional height and at least second elements that have a second z-directional height. The three-dimensional substrates may also have at least third elements having at least third z-directional heights. The first z-directional height may be greater than the second and third z-directional heights. The second z-directional height may be greater than the third z-directional height. The first z-directional height may be less than the second z-directional height with the first z-directional height being greater than the third z-directional height. The substrates may also have a plurality of apertures. Owing to such structures, fluids may be quickly moved away from the skin of a wearer, leaving primarily the first elements (or highest elements) having the first z-directional heights contacting the skin of the wearer, thereby making the wearer feel dryer. The fluids may flow via gravity and/or via capillary gradients into the second elements (or lower or mid level elements) having the second z-directional heights, the third elements (or lowest elements) having the third z-directional heights, and/or into and through the apertures, so that the fluids may be absorbed into the absorbent articles. By providing the three-dimensional substrates of the present disclosure, fluid/skin contact and the time that fluids are in contact with the skin of a wearer may be reduced. Further, the first elements (or highest elements) having the first z-directional heights may act as a spacer between the fluids and the skin of the wearer while the fluids are being absorbed into the absorbent article.

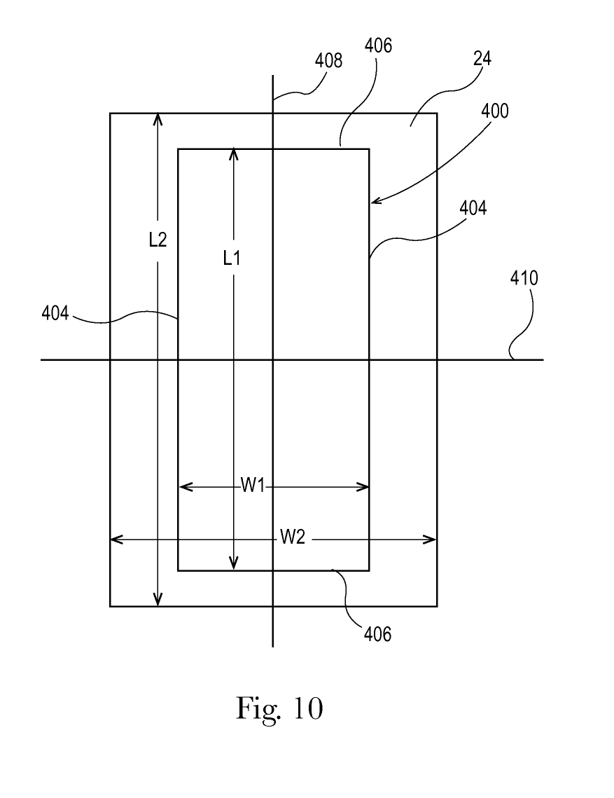

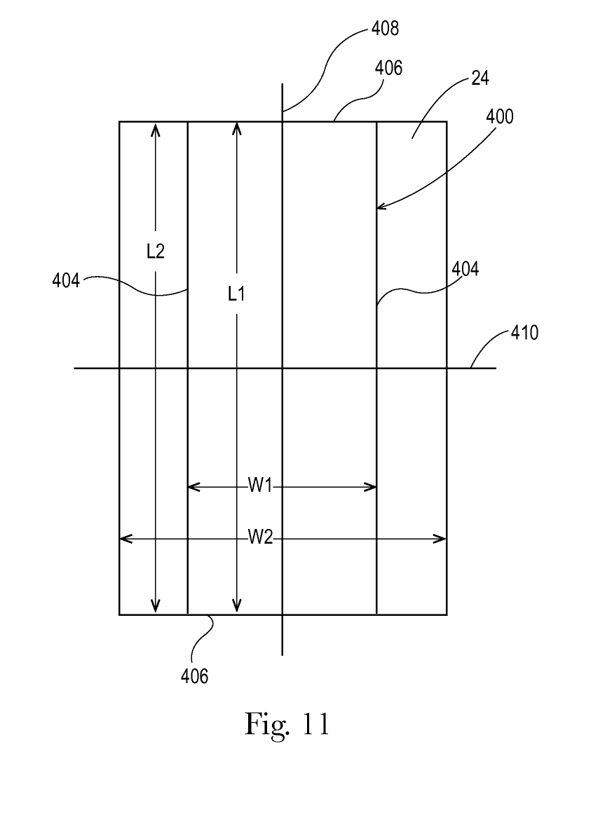

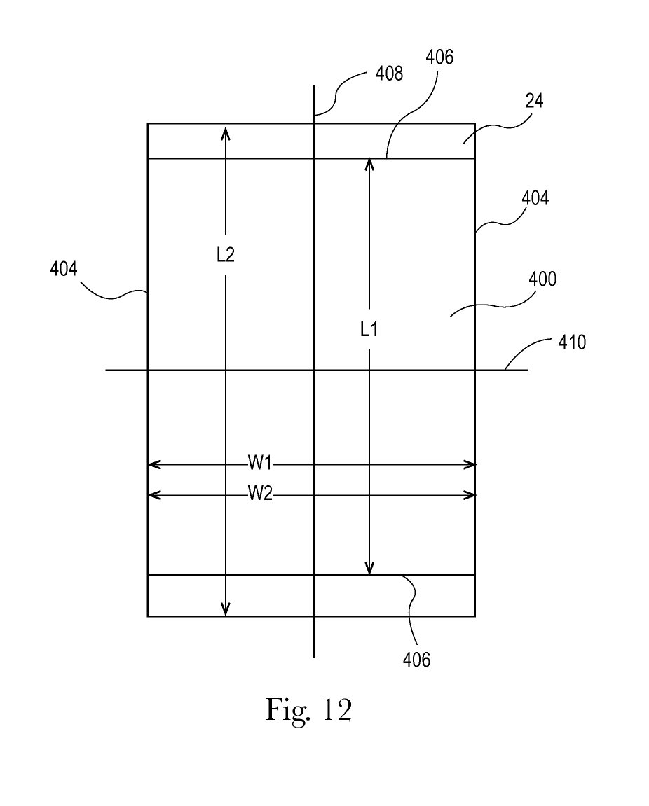

In one form, referring to FIGS. 10-12, a three-dimensional, liquid permeable substrate 400 or other liquid permeable substrates described herein may comprise a patch or strip positioned on and/or joined to a topsheet of the absorbent article 402. The patch or strip may be bonded to the topsheet, adhesively attached to the topsheet, cold-pressure welded to the topsheet, ultrasonically bonded to the topsheet, or otherwise joined to the topsheet. Alternatively, the liquid permeable substrates of the present disclosure may comprise the topsheet (e.g., topsheet 24), form all of the topsheet, or form a portion of the topsheet. Also, the topsheet 24 may be comprised only of one or more of the liquid permeable substrates of the present disclosure. In any of the various configurations, the liquid permeable substrates of the present disclosure are intended to form at least a portion of the wearer-facing surface of an absorbent article and be in at least partial contact with the skin of a wearer.

Referring again to FIGS. 10-12, the three-dimensional, liquid permeable substrate 400, or other liquid permeable substrates described herein, in a patch or strip form joined to the topsheet 24, may have a cross machine directional width of W1, while the topsheet 24 may have a cross machine directional width of W2. W1 may be less than, the same as, substantially the same as, or greater than (not illustrated) the width W2. The width W1 may also vary or be constant throughout a longitudinal length of the liquid permeable substrates. Still referring to FIGS. 10-12, the liquid permeable substrate 400, or other liquid permeable substrates described herein, in a patch or strip form, may have a machine directional length of L1, while the topsheet 24 may have a machine directional length of L2. L1 may be less than, the same as, substantially the same as, or greater than (not illustrated) the length L2. The length L1 may vary or be constant across the width W1 of the liquid permeable substrates. Although not illustrated in FIGS. 10-12, the lengths and widths of the topsheet 24 and the liquid permeable substrates may be the same, or substantially the same.

Although the patch or strip of the liquid permeable substrate 400 is illustrated as being rectangular in FIGS. 10-12, the liquid permeable substrates of the present disclosure may also have any other suitable shapes, such a front/back profiled shape (i.e., wider in the front, wider in the back, and/or narrower in the crotch), a square shape, an ovate shape, or other suitable shape. The side edges 404 and/or the end edge 406 of the liquid permeable substrate 400 may have one or more arcuate portions, designs, and/or shapes cut out from them to provide an aesthetically pleasing look to the liquid permeable substrate 400. One side edge 404 may be symmetrical or asymmetrical to another side edge 404 about a longitudinal axis, 408, of the topsheet 24. Likewise, one end edge 406 may be symmetrical or asymmetrical to another side edge 406 about a lateral axis, 410 of the topsheet 24.

The liquid permeable substrates of the present disclosure may comprise one or more layers. If more than one layer is provided, the layers may be joined together or attached to each other through mechanical bonding, hydroentangling, embossing, adhesive bonding, pressure bonding, heat bonding, or by other methods of joining to form a multilayer substrate. The first layer may comprise one or more hydrophilic materials, or may be fully hydrophilic, and the second layer may comprise one or more hydrophilic materials, or may be fully hydrophilic. The first and second layers may have different degrees of hydrophilicity. Alternatively, one of the layers may be hydrophobic or at least more hydrophobic than the other layer. The first layer may be used as a portion of, or all of, the wearer-facing surface of the absorbent article.

The first layer may comprise a plurality of first fibers and/or filaments (hereafter together referred to as fibers). The plurality of first fibers may comprise fibers that are the same, substantially the same, or different in size, shape, composition, denier, fiber diameter, fiber length, and/or weight. The first layer may also comprise one or more layers, each comprising different or the same fibers. The second layer may comprise a plurality of second fibers. The plurality of second fibers may comprise fibers that are the same, substantially the same, or different in size, shape, composition, denier, fiber diameter, fiber length, and/or weight. The plurality of first fibers may be the same as, substantially the same as, or different than the plurality of second fibers. The second layer may also comprise one or more layers, each comprising different or the same fibers. Additional layers may have the same or different configurations.

The first layer, or wearer-facing layer if provided on an absorbent article, may comprise spunbond fibers, polyester fibers, polypropylene fibers, other polyolefin fibers, carded fibers, bicomponent fibers (e.g., PE/PET bicomponent fibers), and/or other suitable fibers. The second layer, or garment-facing layer if provided on an absorbent article, may comprise spunbond fibers, carded fibers, polyester fibers, polypropylene fibers, other polyolefin fibers, bicomponent fibers, and/or other suitable fibers. In other forms, the first layer (wearer-facing) may comprise the spunbond fibers and the second layer (garment facing) may comprise the carded fibers. The first layer may be joined to the second layer to form the liquid permeable substrate.

At least some of, or all of, the fibers of the first layer may have a different denier and fiber diameter than at least some of, or all of, the fibers of the second layer. As an example, the polyester fibers of the first layer may have a different denier and fiber diameter than the polypropylene fibers of the second layer. The denier of the fibers of the first layer may be smaller than the denier of the fibers of the second layer. As an example, the fibers of the first layer may have a denier in the range of about 0.1 to about 3, about 0.5 to about 2, about 0.75 to about 2, about 0.8 to about 2, about 0.9 to about 1.5, about 0.9, about 1, about 1.1, about 1.2, or about 1.3, specifically reciting all 0.1 denier increments within the specified ranges and all ranges formed therein or thereby. The fibers of the second layer may have a denier in the range of about 0.9 to about 4, about 1 to about 3, about 1.5 to about 2.5, about 1.7 to about 2.3, about 1.9, about 2, or about 2.1, specifically reciting all 0.1 denier increments within the specified ranges and all ranges formed therein or thereby. Denier is defined as the mass in grams per 9000 meters of a fiber length. Having the first layer comprising fibers having a smaller denier than the denier of the fibers of the second layer may allow the wearer-facing surface to remain dryer. The pores of the first layer may be larger than the pores of the second layer. The diameters of the fibers in the first layer may be in the range of about 5 μm to about 20 μm, about 8 μm to about 15 μm, about 9 μm to about 13 μm, about 10 to about 12 μm, or may be about 11 μm. The diameters of the fibers in the second layer may be in the range of about 10 μm to about 30 μm, about 12 μm to about 25 μm, about 15 μm to about 22 μm, about 16 μm to about 20 μm, or may be about 18 μm. In one form, the diameter of the fibers in the first layer may be smaller than the diameter of the fibers in the second layer.

The fibers in the first and second layers may also comprise any other suitable types of fibers, such as other polyolefins, polylactic acid, thermoplastic starch-containing sustainable resins, other sustainable resins, bio-PE fibers, bio-PP fibers, bio-PET fibers, viscose fibers, rayon fibers, or other suitable fibers, for example. These fibers may have any suitable deniers or denier ranges and/or fiber lengths or fiber length ranges. The fibers in the first and second layers may be treated with a hydrophilic agent, such as a surfactant, to cause the fibers to become hydrophilic or at least less hydrophobic. Alternatively, the substrates, or portions thereof, may be treated with the hydrophilic agent to cause the fibers to become hydrophilic or at least less hydrophobic.

The first layer may have a basis weight in the range of about 10 gsm to about 50 gsm, about 15 gsm to about 45 gsm, about 20 gsm to about 40 gsm, about 25 gsm to about 35 gsm, or about 30 gsm. The second layer may have a basis weight in the range of about 3 gsm to about 25 gsm, about 5 gsm to about 25 gsm, about 5 gsm to about 20 gsm, about 5 gsm to about 15 gsm, about 5 gsm, about 10 gsm, or about 15 gsm. The basis weight of the liquid permeable substrate (first and at least second layers or three or more layers) may be in the range of about 15 gsm to about 75 gsm, about 20 gsm to about 60 gsm, about 25 gsm to about 50 gsm, about 35 gsm to about 45 gsm, about 30 gsm to about 40 gsm, about 35 gsm, about 40 gsm, or about 45 gsm. All other suitable basis weight ranges for the first and second layers and the combined substrate are within the scope of the present disclosure. Accordingly, the basis weight of the layers and the substrates may be designed for specific product requirements. In general, the liquid permeable substrates of the present disclosure may have a greater basis weight than the topsheet 24 (if provided).

Specifically recited herein are all 0.1 gsm increments within the above-specified ranges of basis weight and all ranges formed therein or thereby.

The liquid permeable substrates of the present disclosure may also form a portion of, or all of, the outer cover 23 which is joined to at least a portion of the backsheet 25. In other instances, the outer cover 23 may comprise a pattern (e.g., embossed pattern, printed pattern) and/or three-dimensional structure that is the same as, or similar in appearance to, the liquid permeable substrates of the present disclosure. In general, the appearance of at least a portion of a liquid permeable substrate on the wearer-facing surface may match, or substantially match, at least a portion of the outer cover 23 or another portion of absorbent article.

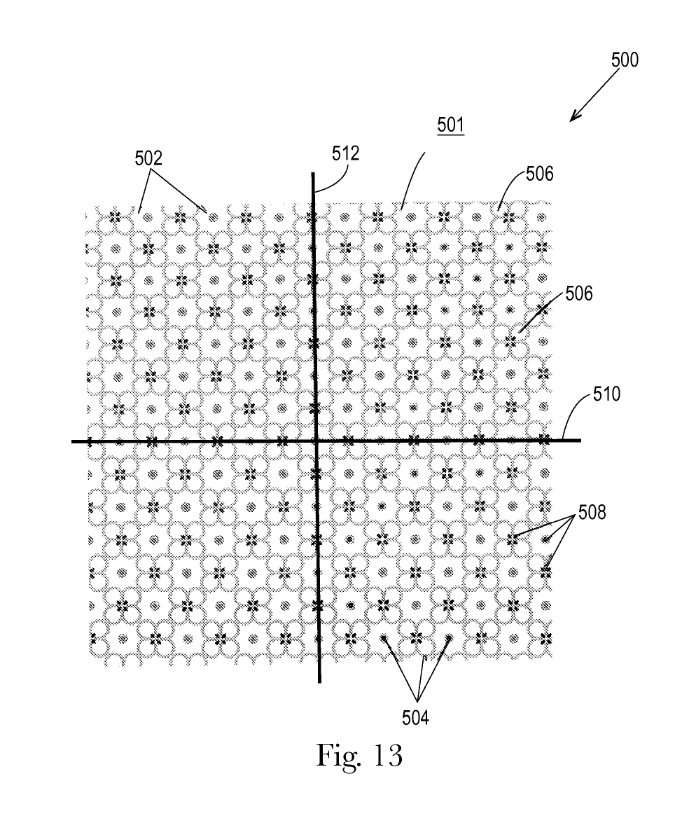

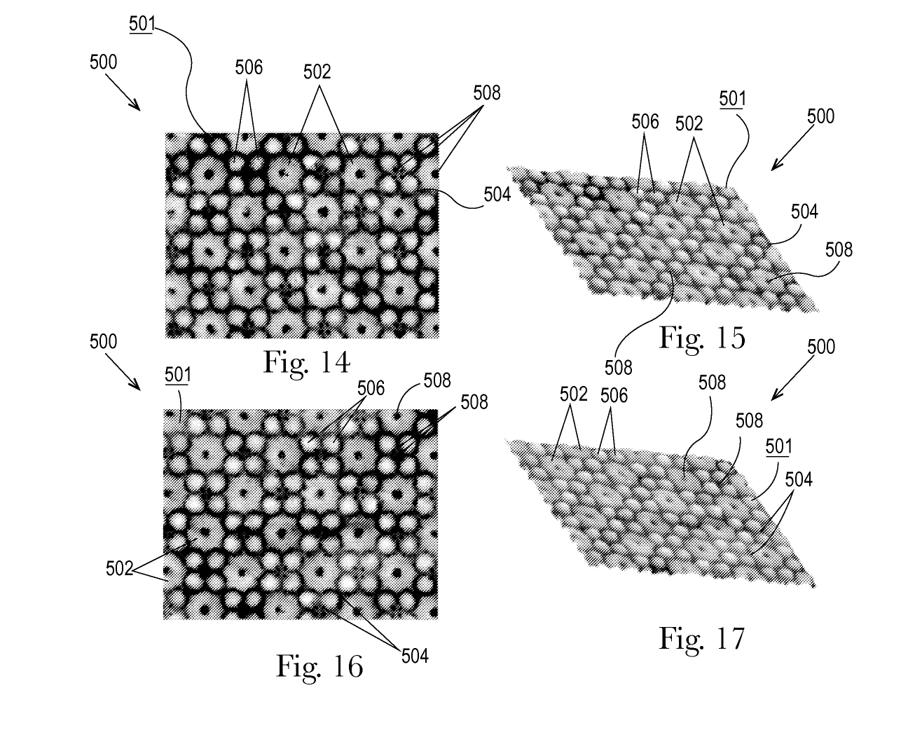

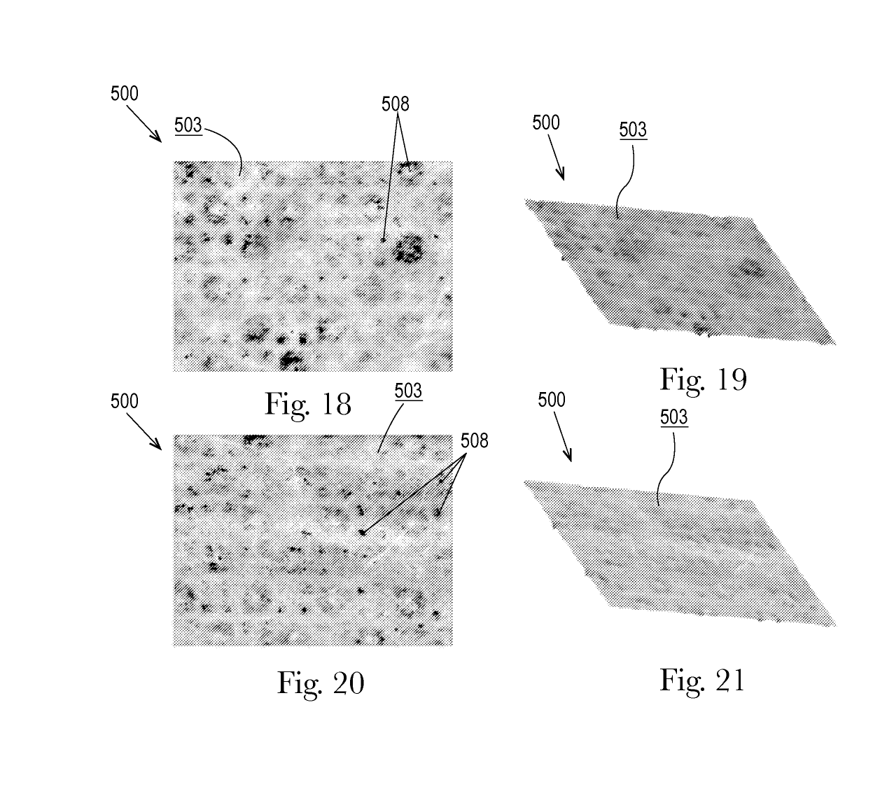

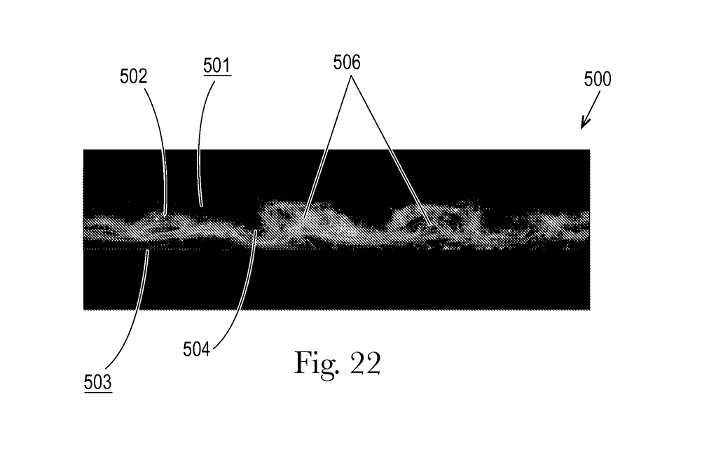

FIG. 13 is a top view of an illustration of a first three-dimensional, liquid permeable substrate 500, wearer-facing surface facing the viewer. FIG. 14 is a front view of a portion of the first liquid permeable substrate 500, wearer-facing surface facing the viewer. FIG. 15 is a perspective view of the portion of the first liquid permeable substrate 500 of FIG. 14. FIG. 16 is another front view, taken from a different portion of the first liquid permeable substrate 500 of FIG. 14, wearer-facing surface facing the viewer. FIG. 17 is a perspective view of the different portion of the first liquid permeable substrate 500 of FIG. 16. FIG. 18 is a rear view of a portion of the first liquid permeable substrate 500, garment-facing surface facing the viewer. FIG. 19 is a perspective view of the portion of the first liquid permeable substrate 500 of FIG. 18. FIG. 20 is another rear view, taken from a different portion of the first liquid permeable substrate 500, garment-facing surface facing the viewer. FIG. 21 is a perspective view of the different portion of the first liquid permeable substrate 500 of FIG. 20. FIG. 22 is a cross-sectional view of the first liquid permeable substrate 500.

Referring generally to FIGS. 13-17 and 22, the first three-dimensional, liquid permeable substrate 500 may comprise a first surface 501 comprising a plurality of first projections 502, a plurality of bridge portions 504, and a plurality of second projections 506. The first substrate 500 may also comprise a plurality of apertures 508. In FIG. 13, the second projections 506 are represented by the white areas, the bridge portions 504 are represented by the dark gray areas, the first projections 502 are represented by the light grey areas, and the apertures 508 are represented by the black areas. A majority of, or all of, the plurality of second projections 506 may have a first z-directional height, according to the according to the Second Projection Height Test described herein. The first z-directional height may be in the range of about 500 μm to about 2100 μm, about 600 μm to about 1900 μm, about 750 μm to about 1750 μm, about 900 μm to about 1600 μm, about 1000 μm to about 1600 μm, about 1100 μm to about 1550 μm, about 1150 μm to about 1500 μm, about 1175 μm to about 1475 μm, about 1200 μm to about 1500 μm, about 1300 μm to about 1450 μm, about 1320 μm, about 1325 μm, or about 1330 μm, specifically reciting all 1 μm increments within the above-specified ranges and all ranges formed therein or thereby. A majority of, or all of, the plurality of first projections 502 may have a second z-directional height, according to the First Projection Height Test described herein. The second z-directional height may be in the range of about 400 μm to about 1600 μm, about 500 μm to about 1400 μm, about 600 μm to about 1200 μm, about 700 μm to about 1100 μm, about 725 μm to about 1050 μm, about 730 μm to about 1025 μm, about 800 μm to about 950 μm, about 850 μm to about 950 μm, about 870 μm to about 900 μm, about 870 μm, about 875 μm, about 880 μm, or about 885 μm, specifically reciting all 1 μm increments within the above-specified ranges and all ranges formed therein or thereby. A majority of, or all of, the plurality of bridge portions 504 may have a third z-directional height, according to the Bridge Portion Height Test described herein. The third z-directional height may be in the range of about 100 μm to about 1100 μm, about 200 μm to about 800 μm, about 250 μm to about 700 μm, about 300 μm to about 650 μm, about 350 μm to about 600 μm, about 375 μm to about 550 μm, about 400 μm to about 525 μm, about 450 μm to about 500 μm, about 470 μm, about 475 μm, about 480 μm, specifically reciting all 1 μm increments within the above-specified ranges and all ranges formed therein or thereby. The first z-directional height may be greater than the second and third z-directional heights. The second z-directional height may be greater than or less than the third z-directional height. The bridge portions 504 may comprise areas of the substrate that are unformed. Stated another way, the bridge portions 504 may have a z-directional height that is the same as the web used to form the substrate, but prior to formation.

The first liquid permeable substrate 500 may have an overall substrate z-directional height. The overall substrate z-directional height is measured according to the Overall Substrate Height Test for Substrates with First and Second Projections described herein. The overall substrate z-directional height may be in the range of about 750 μm to about 3000 μm, about 1000 nm to about 2500 μm, about 1250 μm to about 2250 μm, about 1500 μm to about 2250 μm, about 1500 μm to about 2100 μm, about 1525 μm to about 2075 μm, about 1600 μm to about 2000 μm, about 1700 μm to about 1900 μm, about 1750 μm to about 1850 μm, or about 1800 μm, specifically reciting all 1 μm increments within the above-specified ranges and all ranges formed therein or thereby. At least a majority of the apertures 508 may be at least partially surrounded, or fully surrounded, by bridge portions 504.

As can be seen in an example first liquid permeable substrate 500 of FIGS. 18-21, a second surface 503 of the first substrate 500 may be generally flat with the apertures extending therethrough.

In alternative forms, the second surface may comprise a three-dimensional texture that is the same as, similar to, or different than, the three-dimensional texture of the first surface 501. The texture of the second surface may be function of the process of producing the substrate. For example, if the substrate is formed through hydroentanglement and/or other water forming technologies, the second surface may be generally flat. This paragraph applies to the other liquid permeable substrates described herein.

The first surface 501 of the first liquid permeable substrate 500 may have a geometrical roughness value in the range of about 3 to about 5, about 3.5 to about 4.5, about 3.8 to about 4.3, about 3.9, about 4, about 4.09, about 4.1, or about 4.2, according to the Descriptive Analysis Roughness Test described herein, specifically reciting all 0.1 increments within the above-specified ranges and all ranges formed therein or thereby. The first surface 501 of the first liquid permeable substrate 500 may have a coefficient of friction the range of about 0.25 to about 0.4, about 0.28 to about 0.35, about 0.3 to about 0.34, about 0.30, about 0.31, about 0.32, about 0.33, or about 0.34, according to the Descriptive Analysis Roughness Test described herein, specifically reciting all 0.01 increments within the above-specified ranges and all ranges formed therein or thereby. The first surface 501 of the first liquid permeable substrate 500 may have a slip stick value in the range of about 0.005 to about 0.025, about 0.008 to about 0.019, about 0.009 to about 0.015, about 0.01, about 0.011, about 0.012, about 0.013, or about 0.014, according to the Descriptive Analysis Roughness Test described herein, specifically reciting all 0.001 increments within the above-specified ranges and all ranges formed therein or thereby.

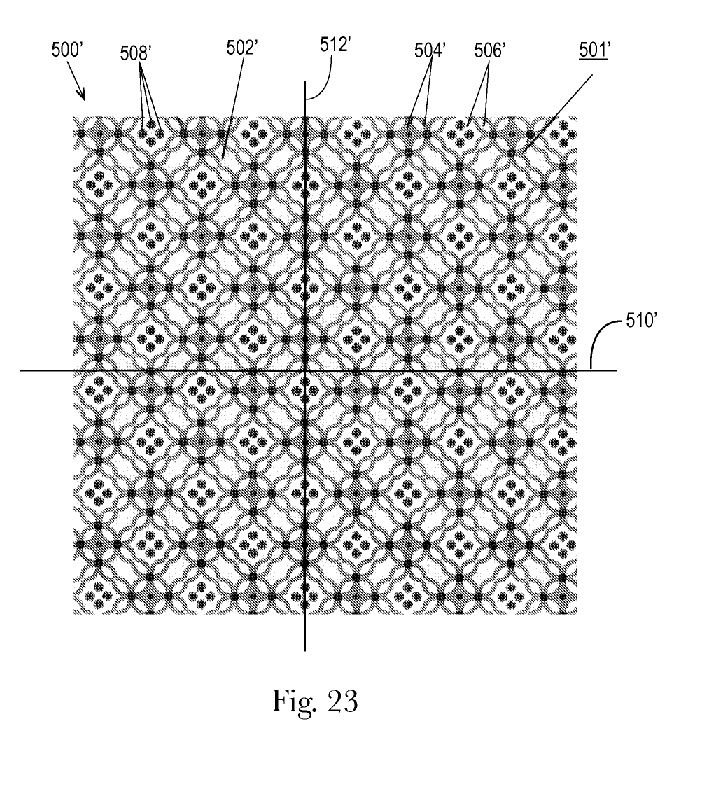

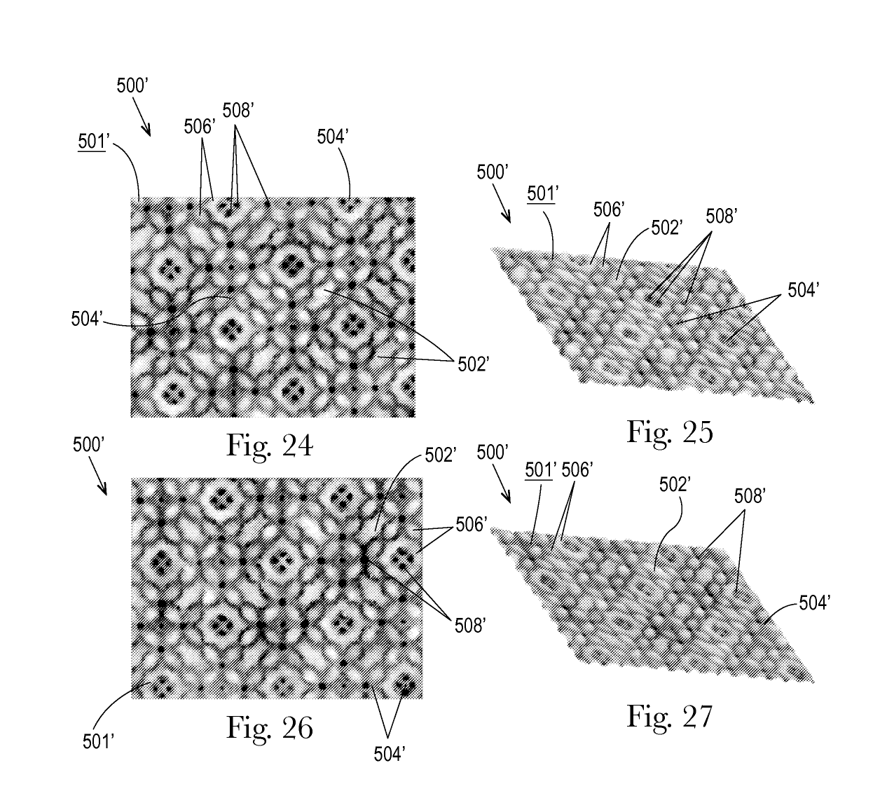

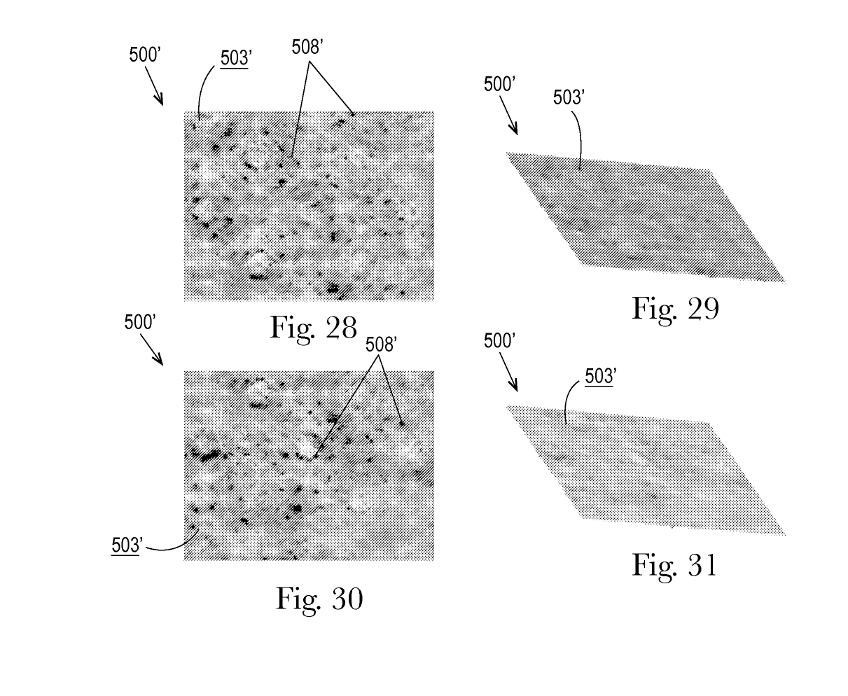

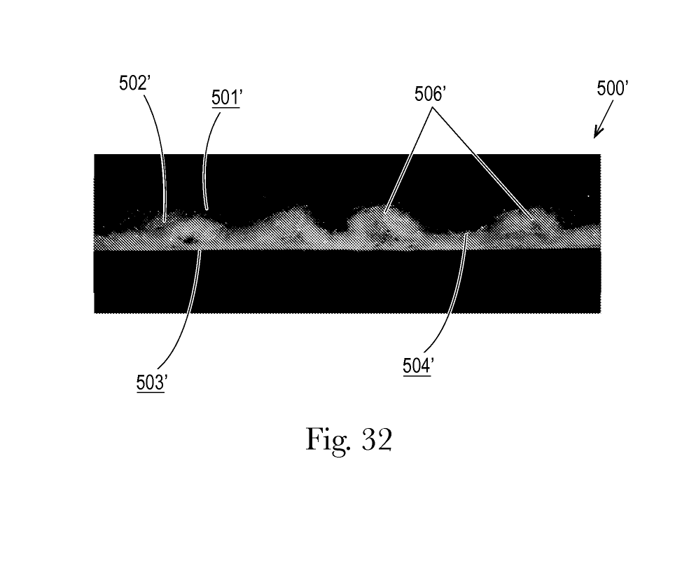

FIG. 23 is a top view of an illustration of a second three-dimensional, liquid permeable substrate 500′, wearer-facing surface facing the viewer. FIG. 24 is a front view of a portion of the second liquid permeable substrate 500′, wearer-facing surface facing the viewer. FIG. 25 is a perspective view of the portion of the second liquid permeable substrate 500′ of FIG. 24. FIG. 26 is another front view, taken from a different portion of the second liquid permeable substrate 500′ of FIG. 24, wearer-facing surface facing the viewer. FIG. 27 is a perspective view of the different portion of the second liquid permeable substrate 500′ of FIG. 26. FIG. 28 is a rear view of a portion of the second liquid permeable substrate 500′, garment-facing surface facing the viewer. FIG. 29 is a perspective view of the portion of the second liquid permeable substrate 500′ of FIG. 28. FIG. 30 is another rear view, taken from a different portion of the second liquid permeable substrate 500′, garment-facing surface facing the viewer. FIG. 31 is a perspective view of the different portion of the second liquid permeable substrate 500′ of FIG. 30. FIG. 32 is a cross-sectional view of the second liquid permeable substrate 500′.

Referring generally to FIGS. 23-27 and 32, the second three-dimensional, liquid permeable substrate 500′ may comprise a first surface 501′ comprising a plurality of first projections 502′, a plurality of bridge portions 504′, and a plurality of second projections 506′. The substrate second 500′ may also comprise a plurality of apertures 508′. In FIG. 23, the second projections 506′ are represented by the white areas, the bridge portions 504′ are represented by the dark gray areas, the first projections 502′ are represented by the light grey areas, and the apertures 508′ are represented by the black areas. A majority of, or all of, the plurality of second projections 506′ may have a first z-directional height, according to the Second Projection Height Test described herein. The first z-directional height may be in the range of about 300 μm to about 1800 μm, about 400 μm to about 1600 μm, about 400 μm to about 1400 μm, about 500 μm to about 1300 μm, about 600 μm to about 1200 μm, about 700 μm to about 1100 μm, about 800 μm to about 1000 μm, about 850 μm to about 950 μm, or about 900 μm, specifically reciting all 1 μm increments within the above-specified ranges and all ranges formed therein or thereby. A majority of, or all of, the plurality of first projections 502′ may have a second z-directional height, according to the First Projection Height Test described herein. The second z-directional height may be in the range of about 300 μm to about 1800 μm, about 500 μm to about 1600 μm, about 700 μm to about 1400 μm, about 750 μm to about 1300 μm, about 800 μm to about 1250 μm, about 810 μm to about 1240 μm, about 900 μm to about 1100 μm, about 950 μm to about 1050 μm, about 1000 μm to about 1050 μm, or about 1025 μm, specifically reciting all 1 μm increments within the above-specified ranges and all ranges formed therein or thereby. A majority of, or all of, the plurality of bridge portions 504′ may have a third z-directional height, according to the Bridge Portion Height Test described herein. The bridge portion z-directional height may be in the range of about 150 μm to about 2000 μm, about 200 μm to about 1800 μm, about 300 μm to about 1300 μm, about 400 μm to about 1200 μm, about 500 μm to about 1100 μm, about 550 μm to about 1025 μm, about 600 μm to about 900 μm, about 700 μm to about 850 μm, about 750 μm to about 800 μm, about 775 μm, specifically reciting all 1 μm increments within the above-specified ranges and all ranges formed therein or thereby. The first z-directional height may be greater than the second and third z-directional heights. The second z-directional height may be greater than or less than the third z-directional height. The bridge portions 504′ may comprise areas of the substrate that are unformed. Stated another way, the bridge portions 504′ may have a z-directional height that is the same as the web used to form the substrate, but prior to formation.

The second liquid permeable substrate 500′ may have an overall substrate z-directional height. The overall substrate height is measured according to the Overall Substrate Height Test for Substrates with First and Second Projections described herein. The overall substrate z-directional height may be in the range of about 500 μm to about 3500 μm, about 750 μm to about 3000 μm, about 1000 μm to about 2750 μm, about 1200 μm to about 2500 μm, about 1300 μm to about 2350 μm, about 1350 μm to about 2300 μm, about 1350 μm to about 2250 μm, or about 1500 μm to about 2000 μm, specifically reciting all 1 μm increments within the above-specified ranges and all ranges formed therein or thereby. At least a majority of the apertures 508′ may be at least partially surrounded, or fully surrounded, by bridge portions 504′.

As can be seen in an example second liquid permeable substrate 500′ of FIGS. 28-31, a second surface 503′ of the substrate may be generally flat with the apertures 508′ extending therethrough.

The first surface 501′ of the second liquid permeable substrate 500′ may have a geometrical roughness value in the range of about 3.5 to about 6.5, about 4.0 to about 6, about 4.5 to about 5.7, about 5.0 to about 6.0, about 5.0 to about 5.5, about 5, about 5.1, about 5.2, about 5.3, or about 5.4, according to the Descriptive Analysis Roughness Test described herein, specifically reciting all 0.1 increments within the above-specified ranges and all ranges formed therein or thereby. The first surface 501′ of the second liquid permeable substrate 500′ may have a coefficient of friction the range of about 0.25 to about 0.4, about 0.28 to about 0.35, about 0.3 to about 0.34, about 0.31, about 0.32, about 0.33, about 0.34, or about 0.35, according to the Descriptive Analysis Roughness Test described herein, specifically reciting all 0.01 increments within the above-specified ranges and all ranges formed therein or thereby. The first surface 501′ of the second liquid permeable substrate 500′ may have a slip stick value in the range of about 0.005 to about 0.025, about 0.007 to about 0.019, about 0.07 to about 0.014, about 0.008, about 0.009, about 0.01, about 0.011, about 0.012, or about 0.013, according to the Descriptive Analysis Roughness Test described herein, specifically reciting all 0.001 increments within the above-specified ranges and all ranges formed therein or thereby.

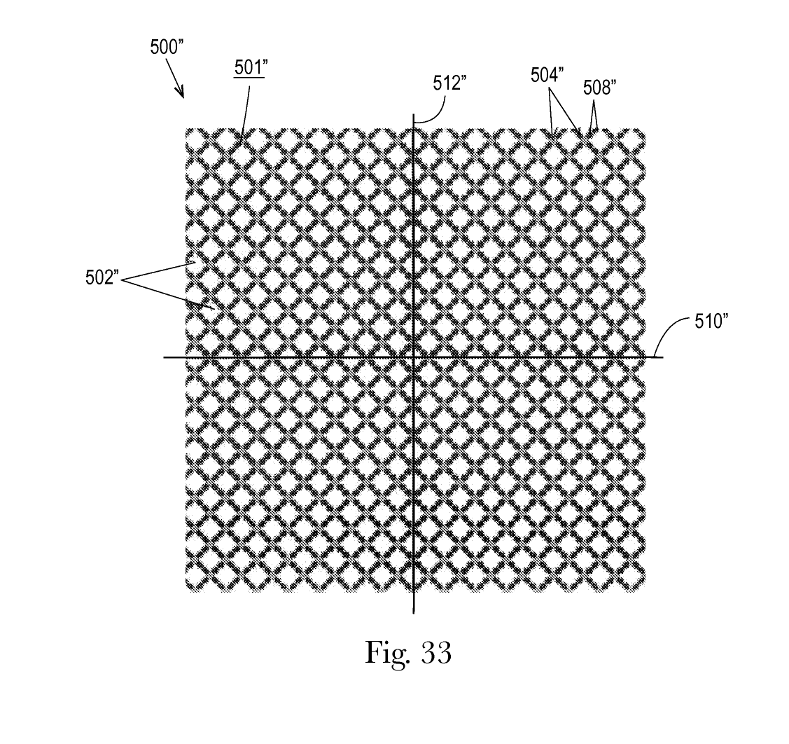

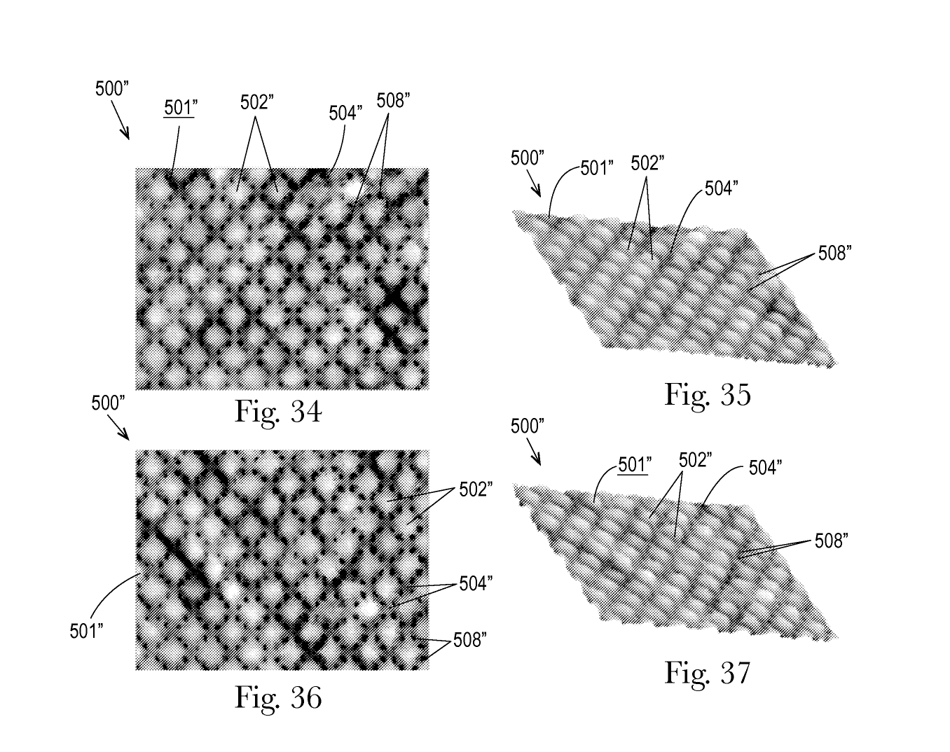

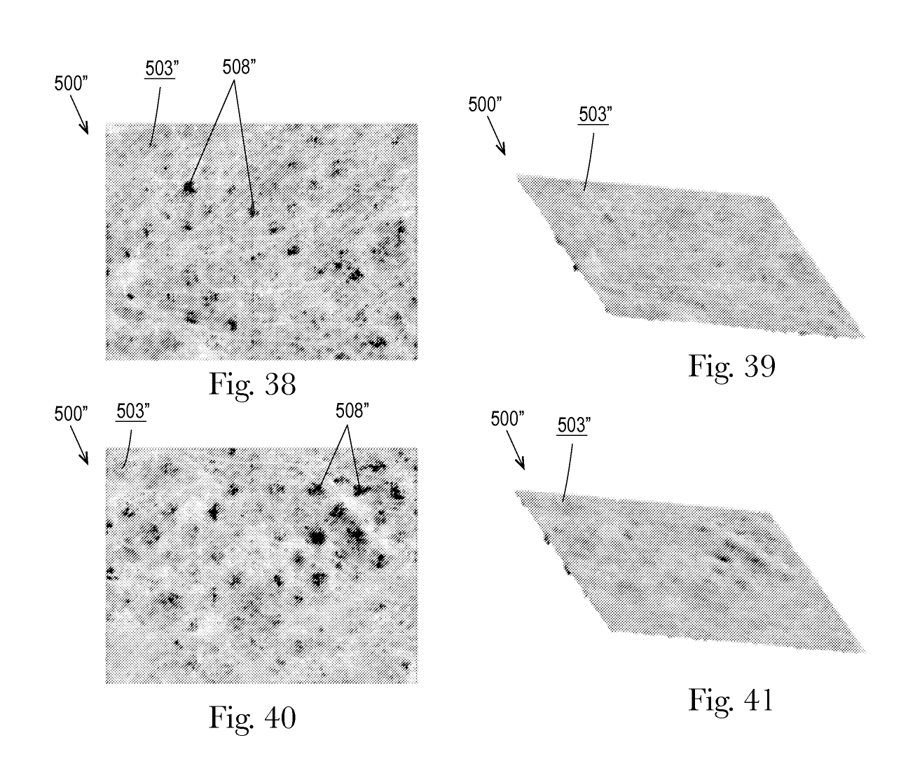

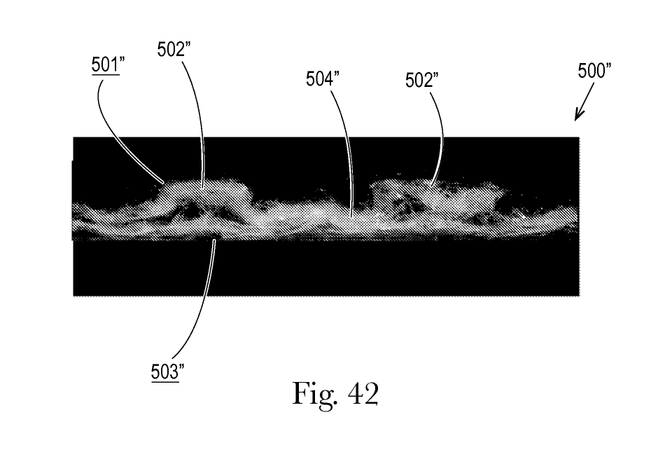

FIG. 33 is a top view of an illustration of a third three-dimensional, liquid permeable substrate 500″, wearer-facing surface facing the viewer. FIG. 34 is a front view of a portion of the third liquid permeable substrate 500″, wearer-facing surface facing the viewer. FIG. 35 is a perspective view of the portion of the third liquid permeable substrate 500″ of FIG. 34. FIG. 36 is another front view, taken from a different portion of the third liquid permeable substrate 500″ of FIG. 34, wearer-facing surface facing the viewer. FIG. 37 is a perspective view of the different portion of the third liquid permeable substrate 500″ of FIG. 36. FIG. 38 is a rear view of a portion of the third liquid permeable substrate 500″, garment-facing surface facing the viewer. FIG. 39 is a perspective view of the portion of the third liquid permeable substrate 500″ of FIG. 38. FIG. 40 is another rear view, taken from a different portion of the third liquid permeable substrate 500″, garment-facing surface facing the viewer. FIG. 41 is a perspective view of the different portion of the third liquid permeable substrate 500″ of FIG. 40. FIG. 42 is a cross-sectional view of the third liquid permeable substrate 500″.

Referring generally to FIGS. 33-37 and 42, the third three-dimensional, liquid permeable substrate 500″ may comprise a first surface 501″ comprising a plurality of projections 502″ and a plurality of bridge portions 504″. The third substrate 500″ may also comprise a plurality of apertures 508″. In FIG. 33, the projections 502″ are represented by the white areas, the bridge portions 504″ are represented by the dark gray areas, and the apertures 508″ are represented by the black areas. A majority of, or all of, the plurality of projections 502″ may have a first z-directional height, according to the Second Projection Height Test described herein. The first z-directional height may be in the range of about 400 μm to about 2500 μm, about 500 μm to about 2000 μm, about 750 μm to about 1750 μm, about 800 μm to about 1600 μm, about 900 μm to about 1500 μm, about 950 μm to about 1400 μm, about 1000 μm to about 1300 μm, about 1100 μm to about 1250 μm, about 1125 μm to about 1225 μm, about 1150 μm to about 1200 μm, or about 1175 μm, specifically reciting all 1 μm increments within the above-specified ranges and all ranges formed therein or thereby. A majority of, or all of, the plurality of bridge portions 504″ may have a second z-directional height, according to the Bridge Portion Height Test described herein. The bridge portion z-directional height may be in the range of about 100 μm to about 1500 μm, about 300 μm to about 1400 μm, about 400 μm to about 1300 μm, about 500 μm to about 1200 μm, about 550 μm to about 1075 μm, about 700 μm to about 1000 μm, about 750 μm to about 900 μm, about 775 μm to about 850 μm, about 800 μm to about 825 μm, or about 810 μm, specifically reciting all 1 μm increments within the above-specified ranges and all ranges formed therein or thereby. The first z-directional height may be greater than the second z-directional height. The bridge portions 504″ may comprise areas of the substrate that are unformed. Stated another way, the bridge portions 504″ may have a z-directional height that is the same as the web used to form the substrate, but prior to formation.

The third liquid permeable substrate 500″ may have an overall substrate z-directional height. The overall substrate height is measured according to the Overall Substrate Height Test for Substrates with Projections but no Second Projections described herein. The overall substrate z-directional height may be in the range of about 750 μm to about 3500 μm, about 1000 μm to about 3000 μm, about 1200 μm to about 2700 μm, about 1400 μm to about 2600 μm, about 1500 μm to about 2500 μm, or about 1525 μm to about 2450 μm, specifically reciting all 1 μm increments within the above-specified ranges and all ranges formed therein or thereby. At least a majority of, or all of, the apertures 508′ may be at least partially surrounded, or fully surrounded, by bridge portions 504′.

As can be seen in an example third liquid permeable substrate 500″ of FIGS. 38-41, a second surface 503″ of the third substrate 500″ may be generally flat with the apertures 508″ extending therethrough.

The first surface 501″ of the third liquid permeable substrate 500″ may have a geometrical roughness value in the range of about 3.0 to about 6.0, about 3.5 to about 5.5, about 3.5 to about 4.5, about 3.8 to about 4.7, about 3.9, about 4.0, about 4.1, about 4.2, about 4.24, about 4.3, or about 4.4, according to the Descriptive Analysis Roughness Test described herein, specifically reciting all 0.1 increments within the above-specified ranges and all ranges formed therein or thereby. The first surface 501″ of the third liquid permeable substrate 500″ may have a coefficient of friction the range of about 0.20 to about 0.4, about 0.25 to about 0.35, about 0.28 to about 0.32, about 0.28, about 0.29, about 0.30, about 0.31, or about 0.32, according to the Descriptive Analysis Roughness Test described herein, specifically reciting all 0.01 increments within the above-specified ranges and all ranges formed therein or thereby. The first surface 501″ of the second liquid permeable substrate 500″ may have a slip stick value in the range of about 0.005 to about 0.025, about 0.007 to about 0.019, about 0.007 to about 0.015, about 0.009, about 0.010, about 0.011, about 0.012, or about 0.013, according to the Descriptive Analysis Roughness Test described herein, specifically reciting all 0.001 increments within the above-specified ranges and all ranges formed therein or thereby.

The liquid permeable substrates of the present disclosure may be generally symmetrical about the lateral axis 510, 510′, and 510″ (see FIGS. 13, 23, and 33) and/or generally symmetrical about the longitudinal axis 512, 512′, and 512″ (see FIGS. 13, 23, and 33). In other instances, the substrates may not be symmetrical about the lateral axis and/or the longitudinal axis.

The substrates of the present disclosure may comprise one or more colors, dyes, inks, indicias, patterns, embossments, and/or graphics. The colors, dyes, inks, indicias, patterns, embossments, and/or graphics may aid the aesthetic appearance of the substrates.

The substrates of the present disclosure may be used as a portion of, or all of, any suitable products, such as dusters, wipes (wet or dry), makeup removal substrates, paper towels, toilet tissue, facial tissue, medical gowns, surgical substrates, wraps, filtration substrates, or any other suitable products.

### Method of Making the Three-Dimensional Substrates or Absorbent Articles Comprising the Three-Dimensional Substrates

The three-dimensional substrates and absorbent articles comprising three-dimensional substrates of the present disclosure may be made by any suitable methods known in the art. In particular, the articles may be hand-made or industrially produced at high speed.

### Packages

Absorbent articles comprising the liquid permeable substrates of the present disclosure may be placed into packages. The packages may comprise polymeric films and/or other materials. Graphics or indicia relating to properties of the absorbent articles may be formed on, positioned on, and/or placed on outer portions of the packages. Each package may comprise one or more absorbent articles. The absorbent articles may be packed under compression so as to reduce the size or height of the packages, while still providing an adequate amount of absorbent articles per package. By packaging the absorbent articles under compression, caregivers can easily handle and store the packages, while also providing distribution savings to manufacturers.

Accordingly, packages of the absorbent articles comprising the liquid permeable substrates of the present disclosure may have an in-bag stack height of less than about 100 mm, less than about 95 mm, less than about 90 mm, less than about 85 mm, less than about 80 mm, less than about 78 mm, or less than about 76 mm, according to the In-Bag Stack Height Test described herein. Alternatively, packages of the absorbent articles comprising the liquid permeable substrates of the present disclosure may have an in-bag stack height of from about 70 mm to about 100 mm, from about 70 mm to about 95 mm, from about 72 mm to about 85 mm, from about 72 mm to about 80 mm, or from about 74 mm to about 78 mm, specifically reciting all 0.1 mm increments within the specified ranges and all ranges formed therein or thereby, according to the In-Back Stack Height Test described herein. Further details regarding in-back stack height are disclosed in U.S. Pat. No. 8,585,666, to Weisman et al., issued on Nov. 19, 2013.

### Test Methods

### Height Tests

Substrate z-directional heights (of various portions) and overall substrate heights are measured using a GFM MikroCAD Premium instrument commercially available from GFMesstechnik GmbH, Teltow/Berlin, Germany. The GFM MikroCAD Premium instrument includes the following main components: a) a DLP projector with direct digital controlled micro-mirrors; b) a CCD camera with at least a 1600×1200 pixel resolution; c) projection optics adapted to a measuring area of at least 60 mm×45 mm; d) recording optics adapted to a measuring area of at least 60 mm×45 mm; e) a table tripod based on a small hard stone plate; f) a blue LED light source; g) a measuring, control, and evaluation computer running ODSCAD software (version 6.2, or equivalent); and h) calibration plates for lateral (x-y) and vertical (z) calibration available from the vendor.

The GFM MikroCAD Premium system measures the surface height of a sample using the digital micro-mirror pattern fringe projection technique. The result of the analysis is a map of surface height (z-directional or z-axis) versus displacement in the x-y plane. The system has a field of view of 60×45 mm with an x-y pixel resolution of approximately 40 microns. The height resolution is set at 0.5 micron/count, with a height range of +/−15 mm. All testing is performed in a conditioned room maintained at about 23±2° C. and about 50±2% relative humidity.

A steel frame (100 mm square, 1.5 mm thick with an opening 70 mm square) is used to mount the specimen. Take the steel frame and place double-sided adhesive tape on the bottom surface surrounding the interior opening. To obtain a specimen, lay the absorbent article flat on a bench with the wearer-facing surface directed upward. Remove the release paper of the tape, and adhere the steel frame to the topsheet (substrates described herein may only form a portion of the topsheet, e.g., by being positioned on the topsheet—the three-dimensional material is what is sampled) of the absorbent article. Using a razor blade, excise the topsheet from the underling layers of the absorbent article around the outer perimeter of the frame. Carefully remove the specimen such that its longitudinal and lateral extension is maintained. A cryogenic spray (such as Cyto-Freeze, Control Company, Houston Tex.) can be used to remove the topsheet specimen from the underling layers, if necessary. Five replicates obtained from five substantially similar absorbent articles are prepared for analysis.

Calibrate the instrument according to manufacturer's specifications using the calibration plates for lateral (x-y axis) and vertical (z axis) available from the vendor.

Place the steel plate and specimen on the table beneath the camera, with the wearer-facing surface oriented toward the camera. Center the specimen within the camera field of view, so that only the specimen surface is visible in the image. Allow the specimen to lay flat with minimal wrinkles.

Collect a height image (z-direction) of the specimen by following the instrument manufacturer's recommended measurement procedures. Select the Technical Surface/Standard measurement program with the following operating parameters: Utilization of fast picture recording with a 3 frame delay. Dual phaseshifts are used with 1) 16 pixel stripe width with a picture count of 12 and 2) 32 pixel stripe width with a picture count of 8. A full Graycode starting with pixel 2 and ending with pixel 512. After selection of the measurement program, continue to follow the instrument manufacturer's recommended procedures for focusing the measurement system and performing the brightness adjustment. Perform the 3D measurement then save the height image and camera image files.

Load the height image into the analysis portion of the software via the clipboard. The following filtering procedure is then performed on each image: 1) removal of invalid points; 2) removal of peaks (small localized elevations); 3) polynomial filtering of the material part with a rank of n=5, with exclusion of 30% of the peaks and 30% of the valleys from the material part, and 5 cycles.

### First Projection Height Test

Draw a line connecting two first projections, with the line crossing a bridge portion located between the two first projections. Generate a sectional image of the height image along the drawn line. Along the sectional line, measure the vertical height (z-direction) difference between the two first projections and the adjacent valley of the bridge portion. Record the height to the nearest 0.1 μm. Average together 10 different first projection to bridge portion height measures and report this value to the nearest 0.1 μm. This is the first projection height.

### Second Projection Height Test

Draw a line connecting two second projections (or projections if no second projections are provided in the substrate), with the line crossing a bridge portion located between the two second projections (or projections if no second projections are provided in the substrate). Generate a sectional image of the height image along the drawn line. Along the sectional line, measure the vertical height (z-direction) difference between the two second projections (or projections) and the adjacent valley of the bridge portion. Record the height to the nearest 0.1 μm. Average together 10 different second projection to bridge portion height measures and report this value to the nearest 0.1 μm. This is the second projection height (or projection height).

### Bridge Portion Height Test

Draw a line connecting two bridge portions, with the line crossing an aperture in the substrate. Generate a sectional image of the height image along the drawn line. Along the sectional line, measure the vertical height (z-direction) difference between the bridge portions and the bottommost portion of the aperture (i.e., bottom of the substrate). Record the height to the nearest 0.1 μm. Average together 10 different bridge portion to bottommost portion of aperture height measures and report this value to the nearest 0.1 μm. This is the bridge portion height.

Overall Substrate Height Test for Substrates with First and Second Projections

Add the height of the second projections (or highest projections) and the height of the bridge portions together. This should be done with each of the ten measurements from the Second Projection Height Test and the Bridge Portion Height Test. Average together the heights and report this value to the nearest 0.1 μm. This is the overall substrate height for substrates having first and second projections.

Overall Substrate Height Test for Substrates with Projections and No Second Projections

Add the height of the projections and the height of the bridge portions together. This should be done with each of the ten measurements from the Second Projection Height Test and the Bridge Portion Height Test. Average together the heights and report this value to the nearest 0.1 μm. This is the overall substrate height for substrates having projections and no second projections.

### Basis Weight

Basis weight of the three-dimensional substrates may be determined by several available techniques but a simple representative technique involves taking an absorbent article, removing any elastic which may be present and stretching the absorbent article to its full length. A punch die having an area of 45.6 cm² is then used to cut a piece of the substrate forming a topsheet, positioned on the topsheet, or forming a portion of the topsheet (the “topsheet” in this method), from the approximate center of the diaper or absorbent product in a location which avoids to the greatest extent possible any adhesive which may be used to fasten the topsheet to any other layers which may be present and removing the topsheet layer from other layers (using cryogenic spray, such as Cyto-Freeze, Control Company, Houston, Tex. if needed). The sample is then weighed and dividing by the area of the punch die yields the basis weight of the topsheet. Results are reported as a mean of 5 samples to the nearest 0.1 gram per square meter.

### Descriptive Analysis Roughness Method

Surface Geometrical Roughness is measured using a Kawabata Evaluation System KES FB4 Friction tester with Roughness Sensor (available from Kato Tech Co., Japan). The instrument measures both surface friction and geometric roughness simultaneously, but herein only the geometric roughness (SMD value) is reported. All testing is performed at about 23° C.±2 C.° and about 50%±2% relative humidity. Samples are preconditioned at about 23° C.±2 C.° and about 50%±2% relative humidity for 2 hours prior to testing. The instrument is calibrated as per the manufacturer's instructions.

The absorbent article is placed, wearer-facing surface upward, onto a lab bench. The absorbent article's cuffs are clipped with scissors to facilitate the article lying flat. With scissors or a scalpel excise a specimen of the topsheet 20 cm long in the longitudinal direction of the absorbent article and 10 cm wide in the lateral direction of the absorbent article. Care should be taken in removing the specimen as to not distort the dimensions in either the longitudinal or lateral direction. Specimens are collected from a total of five substantially identical absorbent articles.

Turn on the KES FB4. The instrument should be allowed to warm up for at least 10 minutes before use. Set the instrument to a SMD sensitivity of 2×5, a testing velocity of 0.1, and a compression area of 2 cm. The roughness contractor compression (contact force) is adjusted to 10 gf. Place the topsheet specimen on the tester with the wearer-facing surface facing upward and the longitudinal dimension aligned with the test direction of the instrument. Clamp the specimen with an initial tension of 20 gf/cm. Initiate the test. The instrument will automatically take 3 measurements on the specimen. Record the MIU (Coefficient of Friction), MMD (Slip Stick), and SMD (Geometrical Roughness) value from each of the three measurements to the nearest 0.001 micron. Repeat in like fashion for the remaining four specimens.

Report Coefficient of Friction as an average of the 15 recorded values to the nearest 0.01. Report Slip Stick as an average of the 15 recorded values to the nearest 0.001. Report the Geometrical Roughness as an average of the 15 recorded values to the nearest 0.01 micron.

### In-Bag Stack Height Test

The in-bag stack height of a package of the absorbent articles of the present disclosure is determined as follows:

### Equipment

Universal Diaper Packaging Tester (UDPT) (Model #M-ROEL; Machine #MK-1071), including a horizontal sliding plate (horizontal plate that moves up and down in a vertical plane) for adding weights. It is counter-balanced by a suspended weight to assure that no downward force is added from the horizontal sliding plate assembly to the diaper package at all times. The UDPT is available from Matsushita Industry Co. LTD, 7-21-101, Midorigaoka-cho, Ashiya-city, Hyogo JAPAN. Zip code: 659-0014. A 850 g (+/−0.5 g) weight.

### DEFINITIONS

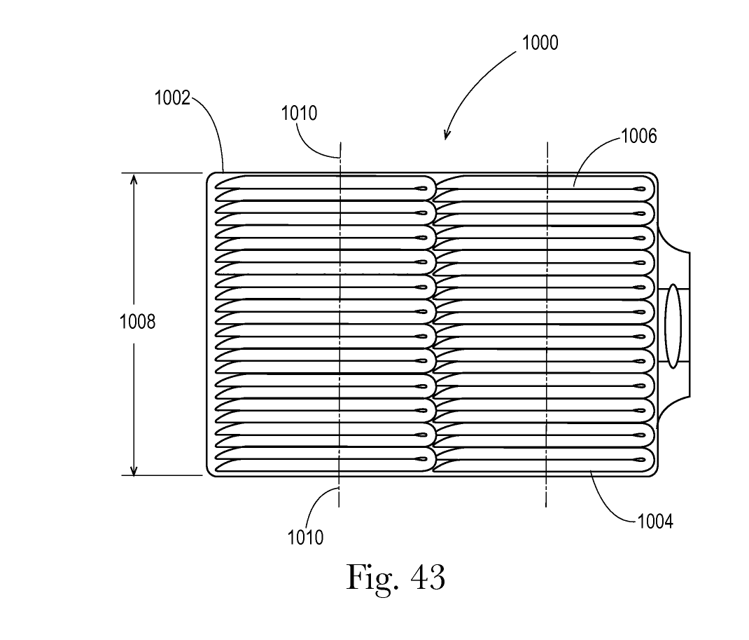

As illustrated in FIG. 43, a package 1000 defines an interior space 1002 and comprises a plurality of absorbent articles 1004. The absorbent articles are in a stack 1006. The package has a package width 1008. The package width 1008 is defined as the maximum distance between the two highest bulging points along the same compression stack axis 1010 of the absorbent article package 1000.

In-Bag Stack Height=(Package Width/Pad Count Per Stack)×10 absorbent articles.

### Apparatus Calibration

Pull down the horizontal sliding plate until its bottom touches the tester base plate.

Set the digital meter located at the side of the horizontal sliding scale to zero mark.
Raise the horizontal sliding plate away from the tester base plate.

### Test Procedure

Put one of the side panels of the absorbent article package along its width standing at the center of the tester base plate.
Make sure the vertical sliding plate (vertical plate that moves left and right in a horizontal plane) is pulled to the right so it does not touch the package being tested.
Add the 850 g weight onto the vertical sliding plate.
Allow the horizontal sliding plate to slide down slowly until its bottom lightly touches desired highest point of the package.
Measure the package width in mm (distance from the top of the base plate to the top of the diaper package).
Record the reading that appears on the digital meter.
Remove the 850 g weight.
Raise the horizontal sliding plate away from the diaper package.
Remove the absorbent article package.

### Calculation/Reporting

Calculate and report the “In-Bag Stack Height”=(Package Width/Pad Count Per Stack)×10.
Report Sample Identification, i.e. complete description of product being tested (product brand name/size).
Report the determined value for each width measurement to the nearest 1 mm. At least five absorbent article packages having the same pad count are measured in this manner for a given product and the in-bag stack height values are aggregated to calculate an average and standard deviation.
Report the Production Date of the measured package (taken from package coding).
Report the Testing Date and Analytical Method used.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any embodiment disclosed or claimed herein or that it alone, or in any combination with any other reference or references, teaches, suggests or discloses any such embodiment. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications may be made without departing from the spirit and scope of the present disclosure. It is therefore intended to cover in the appended claims all such changes and modifications that are within the scope of this disclosure.

What is claimed is:

1. A liquid permeable substrate for an absorbent article, the liquid permeable substrate comprising:
a first layer; and
a second layer joined to the first layer to form a composite;
wherein the composite comprises:
a plurality of apertures defined therein;
a plurality of bridge portions;
a plurality of first projections extending outwardly relative to the bridge portions; and
a plurality of second projections extending outwardly relative to the bridge portions and the first projections, wherein a majority of the plurality of the second projections have a first z-directional height, wherein a majority of the first projections have a second z-directional height, wherein a majority of the plurality of the bridge portion have a third z-directional height, wherein the first z-directional height is greater than the second and third z-directional heights, wherein the second z-directional height is greater than the third z-directional height, and wherein the first z-directional height is in the range of about 1100 μm to about 1600 μm according to the Second Projection Height Test;
wherein the substrate has an overall z-directional height in the range of about 1400 μm to about 2250 μm according to the Overall Substrate Height Test for Substrates with First and Second Projections.

2. The substrate of claim 1, wherein the first layer comprises carded fibers and has a basis weight in the range of about 15 gsm to about 50 gsm, and wherein the first layer forms a wearer-facing surface of the absorbent article.

3. The substrate of claim 1, wherein the second layer comprises spunbond fibers and has a basis weight in the range of about 5 gsm to about 25 gsm.

4. The substrate of claim 1, wherein the first layer comprises polyester fibers, and wherein the second layer comprises polypropylene fibers.

5. The substrate of claim 4, wherein the polyester fibers have a different denier than the polypropylene fibers.

6. The substrate of claim 1, wherein the first layer comprises fibers having a first denier, wherein the second layer comprises fibers having a second denier, and wherein the first denier is different than the second denier.

7. The substrate of claim 1, wherein the third z-directional height is in the range of about 300 μm to about 700 μm according to the Bridge Portion Height Test.

8. The substrate of claim 1, wherein the second z-directional height is in the range of about 600 μm to about 1200 μm according to the First Projection Height Test.

9. The substrate of claim 1, wherein a surface of the first layer positioned opposite to the second layer has a geometrical roughness value in the range of about 3.8 to about 5.5 according to the Descriptive Analysis Roughness Test.

10. The substrate of claim 1, wherein the first layer comprises a hydrophilic material, and wherein the second layer comprises a hydrophilic material.

11. The substrate of claim 1, wherein the first layer or the second layer comprises a surfactant.

12. An absorbent article comprising:
the liquid permeable substrate of claim 1;
a backsheet; and
an absorbent core positioned at least partially intermediate the liquid permeable substrate and the backsheet.

13. The absorbent article of claim 12, wherein the absorbent core is substantially free of airfelt.

14. The absorbent article of claim 13, wherein the absorbent core comprises at least one channel defined therein.

15. The absorbent article of claim 12, comprising an outer cover joined to a portion of the backsheet, wherein the outer cover comprises the liquid permeable substrate of claim 1.

16. The absorbent article of claim 12, comprising an outer cover joined to a portion of the backsheet, wherein the outer cover has the same or a similar appearance as the liquid permeable substrate of claim 1.

17. A package comprising a plurality of the absorbent articles of claim 13, wherein the package has an in-bag stack height of less than about 80 mm, according to the In-Bag Stack Height Test herein.

18. An absorbent article comprising:
a topsheet;
a backsheet;
an absorbent core positioned at least partially intermediate the topsheet and the backsheet; and
the liquid permeable substrate of claim 1 joined to a surface of the topsheet opposite to the absorbent core.

19. The absorbent article of claim 18, wherein the topsheet has a first width measured in a direction parallel to a lateral axis of the topsheet, wherein the liquid permeable substrate has a second width measured in the direction parallel to the lateral axis of the topsheet, and wherein the first width is greater than the second width.

20. The absorbent article of claim 18, wherein the topsheet has a first length measured in a direction parallel to a longitudinal axis of the topsheet, wherein the liquid permeable substrate has a second length measured in the direction parallel to the longitudinal axis of the topsheet, and wherein the first length is greater than the second length.

21. The absorbent article of claim 18, wherein the liquid permeable substrate has a higher basis weight than the topsheet.

22. An absorbent article comprising:
a liquid permeable substrate;
a liquid impermeable backsheet; and
an absorbent core positioned at least partially intermediate the liquid permeable substrate and the liquid impermeable backsheet;
wherein the liquid permeable substrate comprises:
a first layer; and
a second layer joined to the first layer to form a composite;
wherein the composite comprises:
a plurality of apertures defined therein;
a plurality of bridge portions; and
a plurality of projections extending outwardly relative to the bridge portions, wherein a majority of the plurality of projections have a first z-directional height, wherein a majority of the bridge portions have a second z-directional height, and wherein the first z-directional height is greater than the second z-directional height, and wherein the first z-directional height is in the range of about 800 μm to about 1600 μm according to the First Projection Height Test;
wherein the overall z-directional height of the liquid permeable substrate is in the range of about 1400 μm to about 2600 μm according to the Overall Substrate Height Test.

23. The absorbent article of claim 22, wherein the liquid permeable substrate comprises a topsheet.

24. The absorbent article of claim 22, comprising a topsheet comprising a surface positioned opposite from the absorbent core, wherein the liquid permeable substrate is joined to the surface of the topsheet, and wherein the topsheet has a basis weight that is lower than a basis weight of the liquid permeable substrate.

25. The absorbent article of claim 22, wherein the absorbent core is substantially free of airfelt, and wherein the absorbent core comprises two channels and a gasketing cuff.

26. The absorbent article of claim 22, wherein the liquid permeable substrate comprises a surface having a geometrical roughness value in the range of about 3.8 to about 5.5 according to the Descriptive Analysis Roughness Test.

27. A package comprising a plurality of the absorbent articles of claim 22, wherein the package has an in-bag stack height of less than about 78 mm, according to the In-Bag Stack Height Test herein.

28. A liquid permeable substrate for an absorbent article, the liquid permeable substrate comprising:
a first layer; and
a second layer joined to the first layer to form a composite;
wherein the composite comprises:
a plurality of apertures defined therein;
a plurality of bridge portions;
a plurality of first projections extending outwardly relative to the bridge portions; and
a plurality of second projections extending outwardly relative to the bridge portions, wherein a majority of the plurality of the second projections have a first z-directional height, wherein a majority of the first projections have a second z-directional height, wherein a majority of the plurality of the bridge portion have a third z-directional height, wherein the first and second z-directional heights are greater than the third z-directional height, wherein the first z-directional height is different than the second z-directional height, and wherein the second z-directional height is in the range of about 700 μm to about 1400 μm according to the Second Projection Height Test;
wherein the substrate has an overall z-directional height in the range of about 1200 μm to about 2500 μm according to the Overall Substrate Height Test for Substrates with First and Second Projections.
